(19) United States
(12) Patent Application Publication
(10) Pub. No.: US 2025/0339058 A1
(43) Pub. Date: Nov. 6, 2025

(54) Analyte Sensor Applicator

(71) Applicant: Metronom Health, Inc., Laguna Hills, CA (US)

(72) Inventors: Guy Montgomery Heaton, Mission Viejo, CA (US); Neil Martin Becker, Carlsbad, CA (US); Ahramahzd Vanacan Tatavoosian, Mission Viejo, CA (US); Christopher Charles Andrews, Lake Elsinore, CA (US); Troy M. Bremer, Irvine, CA (US)

(21) Appl. No.: 18/840,459

(22) PCT Filed: Feb. 21, 2023
(86) PCT No.: PCT/US2023/013539

§ 371 Date: Aug. 21, 2024

Related U.S. Application Data
(60) Provisional application No. 63/268,363, filed on Feb. 22, 2022.

(51) Int. Cl. A61B 5/145; A61B 5/00; A61B 5/1468

(52) U.S. Cl. CPC A61B 5/14503; A61B 5/14532; A61B 5/14546; A61B 5/1468; A61B 5/6832; A61B 5/6839; A61B 2560/063

(57) ABSTRACT

Different designs of an analyte sensor applicator for applying a wearable analyte monitoring device are disclosed. The analyte sensor applicator includes a housing that holds an analyte sensor assembly, a cam assembly for delivering the analyte sensor assembly, and a skin piercing for inserting a sensor, wherein the skin piercing device is retracted within the housing top after insertion.

### CROSS-REFERENCE TO RELATED APPLICATION

This application claims the benefit of U.S. Provisional Application No. 63/268,363, entitled ANALYTE SENSOR APPLICATOR, filed on Feb. 22, 2022, and which is incorporated by reference herein in its entirety.

### BACKGROUND

### Field

The present disclosure relates generally to applicator for a medical device, and specifically to analyte sensor applicator.

### Description of the Related Art

Analyte tracking and monitoring enable improved monitoring, diagnosis, and treatment of diseases, including diabetes. Existing methods to measure, monitor, and track analyte levels, may include sampling a bodily fluid, preparing the sample for measurement, and estimating the analyte level in the sample. For example, a diabetic patient may prick a finger to obtain a blood sample to measure glucose in a glucose monitoring unit. Such existing methods may be painful or inconvenient for the patient, resulting in lower compliance with physician orders to, for example, take glucose readings at certain times each day or based on patient activity. Effective monitoring, diagnosis, and treatment may benefit from analyte sensors that do not require unpleasant blood draws and/or sample preparation particularly where samples are taken multiple times each day.

Transdermally located sensing elements can be used to provide constant monitoring without requiring unpleasant blood draws to be taken multiple times a day.

### SUMMARY OF THE INVENTION

An analyte monitoring device may include an implantable sensor and/or an implantable portion of the sensor, which may be implanted into the user during monitoring. The analyte monitoring device may further include a wearable analyte monitoring device attached to a user's skin and in connection with the implanted sensor or portion of sensor during monitoring. As a sensor or a portion of the sensor needs to be implanted into a user for measuring, monitoring, and tracking the analyte levels, a sensor implantation system comprising an analyte sensor applicator is necessary. At present, it remains difficult to deploy a sensor or a portion of the sensor into a user's body in a fast, controlled, safe, automatic and less painful way. For example, it is important to take the skin deflection into consideration when designing a sensor implantation system. Skin tissue is an anisotropic, hyper elastic and viscoelastic material. The skin tissue is composed of several layers: epidermis, dermis, hypodermis and the underlying fat and muscle. Layer thickness and composition of the skin tissue may vary with anatomical location, subject physiology and even factors like age and skin hydration. The main structural component of skin is collagen fibers, which are initially unaligned throughout the skin until a load is applied. When skin piercing device penetrates a user's skin, the skin piercing device may impact the surface layer of the skin causing skin deflection. Depending on the types of the user's skin type, the skin deflection may be different in response to the insertion of the skin piercing device. Another difficulty regarding the insertion of the sensor is that after implantation, the sensor may move with the retraction of the sensor delivering device.

In addition, difficulties remain for the manufacture and transportation of the sensor implantable system. For example, the sensor and/or the wearable monitoring device may be damaged during transportation, storage and use of the sensor implantation system. Thus, the sensor implantation system needs to provide good support for the sensor and monitoring device during storage and transportation. Moreover, the manufacturing and assembling of such an implantation system with a wearable monitoring device may remain complicated. For example, the implantation system may include a wearable device containing electronics and an insertable and/or implantable portion. The insertable and/or implantable portion of the implantation system may need to be sterilized while the electronics contained in the wearable device may be damaged during sterilization.

Therefore, substantial need exists to have a fast, controlled and safe sensor implantation system which can minimize the pain and simplify the steps of using the implantation system. In addition, substantial need exists to have a sensor implantation system which can be manufactured with simplified sterilization methods. Substantial need also exists to have a sensor implantation system which can be stored and transported in stable condition without damaging the sensor implantation system or the sensor itself. Some aspects of the present disclosure provide a sensor implantation system and methods thereof to deploy the sensor in a controlled, fast and safe way to minimize the pain.

Some aspects of the present disclosure provide a sensor implantation system which can be stored and transported in a stable condition and manufacture and/or sterilization methods thereof. Some aspects of the present disclosure provide a sensor implantation system including a sterile assembly which can be sterilized before the inclusion of the electronics to the implantation system, such that the sterilization would not damage the electronics.

Some aspects of the present disclosure provide a sensor implantation system including an improved skin-piercing device for deploying and placing the sensor which can avoid damage to the sensor and cause less pain to the user.

In some aspects, an analyte sensor applicator for inserting a sensor of a wearable analyte monitoring device is provided. The analyte sensor applicator may include an applicator housing; a skin-piercing device and an insertable portion of the sensor; a cam assembly within the applicator housing and a piston in contact with the bottom portion of the thrust barrel. The cam assembly may comprise a cylindrical cam comprising a rotational axis and an external cam surface; and a thrust barrel interfacing with the external cam surface, the thrust barrel comprising a bottom portion having a port therethrough for being coupled to the skin-piercing device. The thrust barrel may move linearly between a proximal position and a distal position for inserting the sensor with the skin-piercing device.

In some examples, the thrust barrel interfaces with the external cam surface through one or more grooves. In some examples, upon activation of the cylindrical cam, the piston is pushed to the distal position by the thrust barrel and locked in the distal position and the thrust barrel returns to the proximal position without the piston. In some examples, the skin piercing device is configured to retract to the proximal position independent of the movement of the cylindrical cam. In some examples, the cylindrical cam comprises one or more grooves on the external surface and the thrust barrel comprises one or more pins that ride in the grooves. In some examples, a speed of inserting the sensor is at least partially determined by a pattern of the one or more grooves. In some examples, the thrust barrel comprises one or more grooves on the external cam surface and the cylindrical cam comprises one or more pins that ride in the grooves. In some examples, the analyte sensor applicator further comprises a spring positioned in a cavity of the cylindrical cam, the spring having a spring force that at least partially determines a speed of inserting the sensor. In some examples, the piston comprises a piston base that engages with the wearable analyte monitoring device. In some examples, the housing has a housing bottom and the analyte sensor applicator further comprises a sterile cap key configured to insert into an opening at a distal end of the housing bottom. In some examples, the sterile cap key is provides support to the wearable analyte monitoring device to keep the wearable analyte monitoring device in place. In some examples, the analyte sensor applicator further comprises a sterile cap having a bottom portion and at least one tab, wherein key is configured to loosely engage with the sterile cap. In some examples, the thrust barrel further comprises a magnetic component, the magnetic component magnetically engages with the wearable analyte monitoring device.

In some examples, the applicator comprises an applicator frame having one or more slides on an internal surface of the applicator, the slide is aligned parallel to an axis of the applicator. In some examples, the piston and/or the applicator housing comprises one or more vent holes. In some examples, the piston comprises one or more anti-rotation features, each anti-rotation feature configured to move along one slide.

In some aspects, an implantation system is provided. The implantation system may include the analyte sensor applicator discussed above; and the wearable analyte monitoring device comprising an electronics assembly and sensor components coupled on a proximal side of the wearable analyte monitoring device; and an adhesive component on a distal side of the wearable analyte monitoring device to adhere the wearable analyte monitoring device to a user. In some examples, insertable portions of the skin-piercing device and the sensor project from the distal side.

In another aspect, another design of an analyte sensor applicator for inserting a sensor of a wearable analyte monitoring device is disclosed. The analyte sensor applicator may include a skin-piercing device and an insertable portion of the sensor; a shaft assembly; and a shield in contact with the piston, the shield is configured to be in contact with the wearable analyte monitoring device. In some examples, the shaft assembly comprising: a crank shaft comprising a crankpin; a connecting rod comprising a proximal end in connection with the crank shaft; and a piston in connection with a distal end of the connecting rod, the piston comprising a port for being coupled to the skin-piercing device. In some examples, the connecting rod converts the rotation of the crank shaft into the reciprocating motion of the piston between a proximal position and a distal position for inserting the sensor with the skin-piercing device.

In some examples, the skin-piercing device is configured to retract to the proximal position independent of the movement of the shield. In some examples, the analyte sensor applicator further comprises a spring configured to be release upon activation of the applicator. In some examples, the release of the spring activates the crank shaft. In some examples, upon activation of the crank shaft, the shield is pushed to an insertion position by the piston when the piston reaches the distal position, and the shield is locked in the insertion position while the piston returns to the proximal position without the shield. In some examples, the piston pulls the skin piercing device to the proximal position when the piston returns to the proximal position. In some examples, the shield comprises a plurality of connecting features extending from a base portion of the shield for connecting the shield to an applicator frame, each connecting feature comprising at least one locking features at the proximal end, the applicator frame comprising a plurality holes, each connecting feature configured to slide through one of the plurality of holes until the locking features lock the connecting features to the applicator frame at an insertion position by engaging with a rim of the holes. In some examples, the piston further comprises a magnetic component, the magnetic component magnetically engages with the wearable analyte monitoring device.

In another aspect, another design of an analyte sensor applicator for inserting a sensor of a wearable analyte monitoring device is provided. The analyte sensor applicator may include an actuator comprising a base portion and an arm extending distally from the base portion; an applicator frame configured to receive the arm of the actuator; a piston comprising a port for being coupled to a skin-piercing device; an applicator base is configured to move longitudinally relative to the applicator frame, the applicator base configured to receive the arm of the actuator; an insertion spring configured to drive the applicator base relative to the applicator frame; and a retraction spring. In some examples, in a first position, the arm of the actuator is received by both the applicator frame and the applicator base. In some examples, upon actuation, the analyte sensor applicator transitions from the first position to a second position where the arm of the actuator is released from the applicator base allowing the applicator base and the piston to move relative to the applicator frame.

In some examples, upon actuation, the insertion spring drives the applicator base, the piston and the skin piercing device towards the second position. In some examples, the skin piercing device inserts in a user's skin at the second position. In some examples, the applicator base is configured to be locked at the second position. In some examples, the retraction spring is configured to drive the piston and the skin piercing device away from the second position. In some examples, the piston further comprises a magnetic component, the magnetic component magnetically engages with the wearable analyte monitoring device.

For any of the applicators or wearable analyte monitoring devices described herein, a method of sterilizing an analyte sensor applicator for inserting a sensor of a wearable analyte monitoring device is provided. The method may include sterilizing a base portion of the wearable analyte monitoring device, a skin-piercing device, an insertable portion of the sensor and a sterile cap; assembling the base portion of the wearable analyte monitoring device, the skin-piercing device, the insertable portion of the sensor into a sterile assembly and the cap into a sterile assembly; and after sterilizing the base portion, attaching a removable optical assembly electronics to the base portion of the wearable analyte monitoring device. In some examples, no sterilization is applied to the analyte sensor applicator after attaching the removal optical assembly electronics to the base portion of the wearable analyte monitoring device.

For any of the applicators or wearable analyte monitoring devices described herein, a method of using the analyte sensor applicator discussed above is disclosed. The method may include removing a housing bottom, wherein removing the housing bottom removes a sterile cap attached to the wearable analyte monitoring device covering a tip of the skin-piercing device; placing the applicator on a user's skin; and triggering the applicator to insert the insertable portion of the sensor and place the wearable analyte monitoring device against the user's skin. In some examples, the skin piercing device retracts above the piston after the insertion, such that the user is protected from the skin piercing device.

For any of the applicators described herein, the applicator system may comprise include a housing comprising an opening at a bottom of the housing; a wearable device configured to be housed in the housing; and a key configured to be inserted into the housing through the opening. In some examples, the key is in direct contact with the wearable device and provides support to the wearable device against a component of the applicator system.

For any of the applicators described herein, an applicator system configured to insert a medical device through a skin of a patient is disclosed. The applicator system may comprise a skin piercing device, comprising a proximal portion and a distal portion with one or more cutting edges, the distal portion comprising a partially enclosed body with a lumen extending therethrough, the distal portion configured to house at least a portion of the medical device. In some examples, the distal portion retains the medical device as the medical device is delivered through the skin and releases the medical device upon retraction of the skin piercing device from the skin. In some examples, the distal portion comprises a diameter sized to retain the distal portion without other attachment features. In some examples, the distal portion further comprises a hook configured to attach a medical device to the skin piercing device. In some examples, the medical device comprises a looped distal portion configured to be placed around the hook. In some examples, the looped distal portion comprises protrusions configured to aid in the deployment of the medical device in a user. In some examples, the skin piercing device further comprises a plurality of sliding slots having a long axis along a long axis of the skin piercing device. In some examples, the medical device comprises a sensing area and one or more fasteners, each fastener having an angled surface at a distal end and a concave portion at a proximal end, wherein each sliding slot is configured to receive one fastener. In some examples, the medical device comprises a sensing area, the sensing area having a width smaller than the diameter of the skin piercing device.

For any of the applicators or wearable analyte monitoring devices described herein, an analyte senor for being implanted into a user is provided. The analyte sensor may comprise a sensing area; and one or more fasteners for attaching the analyte sensor to a skin piercing device. In some examples, the one or more fasteners is a looped distal portion of the analyte sensor. In some examples, the looped distal portion of the analyte sensor is configured to be attached to a hook of the skin piercing device. In some examples, the one or more fasteners comprises an angled surface at a distal end and a concave portion at a proximal. In some examples, each of the one or more fasteners is configured to be received in a sliding slot on the skin piercing device having a long axis along a long axis of the skin piercing device.

Any of the systems described above may include an analyte senor for being implanted into a user, the analyte sensor comprising a sensing area, the sensing area configured to be placed inside a skin piercing device.

### BRIEF DESCRIPTION OF THE DRAWINGS

The above-mentioned aspects, as well as other features, aspects, and advantages of the present technology will now be described in connection with various embodiments, with reference to the accompanying drawings. The illustrated embodiments, however, are merely examples and are not intended to be limiting. Throughout the drawings, similar symbols typically identify similar components unless context dictates otherwise. Note that the relative dimensions of the following figures may not be drawn to scale

FIG. 1 illustrates a perspective view of an analyte sensor applicator according to some embodiments.

FIG. 2 illustrates a perspective view of the analyte sensor applicator housing top and housing bottom of the analyte sensor applicator according to FIG. 1.

FIG. 3 illustrates an exploded view of components of the analyte sensor applicator according to FIG. 1.

FIG. 4 illustrates a cross sectional view of the analyte sensor applicator prior to activation according to FIG. 1.

FIG. 5A illustrates a perspective view of a cam according to FIG. 1.

FIG. 5B illustrates a cross-section view of the cam according to FIG. 1.

FIS. 6A and 6B illustrate different cross-section views of the piston 15 according to FIG. 1.

FIG. 7 depicts a cross section view of the applicator according to FIG. 1.

FIG. 8 depicts a cross section view of the applicator according to FIG. 1.

FIG. 9 depicts a perspective view of a cam and a retractor according to FIG. 1.

FIG. 10 illustrates a cross section view of the analyte sensor applicator according to FIG. 1.

FIGS. 11A-11B illustrate cross section views of the analyte sensor applicator according to FIG. 1.

FIG. 12A illustrates a perspective view of an assembled analyte sensor applicator absent housing according to FIG. 1.

FIG. 12B illustrate an exploded view of the components of an analyte sensor applicator absent housing components according to FIG. 1.

FIG. 13 illustrates an exploded view of components of an analyte sensor applicator according to some embodiments.

FIG. 14A illustrates a cross sectional view of the analyte sensor applicator before activation according to FIG. 13.

FIG. 14B illustrates a cross sectional view of the analyte sensor applicator after activation according to FIG. 13.

FIG. 15 illustrates a cross sectional view of the analyte sensor applicator according to FIG. 13.

FIG. 16 illustrates a cross sectional view of the analyte sensor applicator according to FIG. 13.

FIG. 17 illustrates a cross sectional view of the analyte sensor applicator according to FIG. 13.

FIG. 18 illustrates a perspective view of an analyte sensor applicator absent housing according to some embodiments.

FIG. 19 illustrates an exploded view of components of the analyte sensor applicator according to FIG. 18.

FIG. 20 illustrates a cross sectional view of the analyte sensor applicator according to FIG. 18.

FIG. 21 illustrates a cross sectional view of the analyte sensor applicator according to FIG. 18.

FIG. 22 illustrates a cross sectional view of the analyte sensor applicator according to FIG. 18.

FIG. 23 illustrates a perspective view of an analyte sensor applicator absent housing according to some embodiments.

FIG. 24 illustrates an exploded view of components of the analyte sensor applicator according to FIG. 23.

FIG. 25A illustrates a cross sectional view of the applicator frame according to FIG. 23.

FIG. 25B illustrates a perspective view of the applicator base according to FIG. 23.

FIG. 26 illustrates a cross sectional view of an analyte sensor applicator according to FIG. 23.

FIG. 27 illustrates a cross sectional view of an analyte sensor applicator according to FIG. 23.

FIG. 28 illustrates a cross sectional view of an analyte sensor applicator according to FIG. 23.

FIG. 29 illustrates a cross sectional view of an analyte sensor applicator according to FIG. 23.

FIG. 30A illustrates an exploded view of a wearable analyte monitoring device according to some embodiments.

FIG. 30B illustrates a perspective view of a wearable analyte monitoring device according to some embodiments.

FIG. 31 illustrates a cross sectional view of the wearable analyte monitoring device according to FIG. 30B.

FIG. 32 illustrates a cross sectional view of a sterile assembly according to some embodiments.

FIG. 33A illustrates a perspective view of the sterile assembly with removable electronics according to FIG. 32.

FIG. 33B illustrates a perspective view of the sterile assembly w according to FIG. 32.

FIG. 34A illustrates a perspective view of a wearable analyte monitoring device according to some embodiments.

FIG. 34B illustrates a perspective view of an analyte sensor applicator according to some embodiments.

FIG. 35 illustrates a cross section view of an analyte sensor applicator according to some embodiments.

FIG. 36 illustrates a perspective view of a wearable analyte monitoring device according to some embodiments.

FIG. 37 illustrates a cross section view of sterile cap key and housing bottom according to some embodiments.

FIG. 38A illustrates a perspective view of sterile cap key in contact with a wearable analyte monitoring device according to some embodiments.

FIG. 38B illustrates a cross sectional view of the sterile cap key in the analyte sensor applicator according to FIG. 38A.

FIG. 39 illustrates a top-down view of a distal end of a sensor according to some embodiments.

FIG. 40A illustrates a top-down view of a skin piercing device according to some embodiments.

FIG. 40B illustrates a side view of a skin piercing device according to some embodiments.

FIG. 40C illustrates a perspective view of a skin piercing device according to FIG. 40B.

FIG. 41A-41D illustrate perspective views of different designs of distal portion of skin piercing device according to some embodiments.

FIG. 42A illustrates a perspective view of a distal portion of the skin piercing device with an implantable portion of the sensor according to some embodiments.

FIG. 42B illustrates a top-down view of the implantable portion of the sensor according to FIG. 42A.

FIGS. 43A and 43B illustrate a perspective view and a cross sectional view of a distal portion of the skin piercing device with an implantable portion of the sensor respectively according to some embodiments.

FIG. 43C illustrates a top-down view of the implantable portion of the sensor according to FIG. 43A.

### DETAILED DESCRIPTION

Embodiments of the disclosed and described technology relate to sensor implantation systems and methods thereof. The sensor implantation system may comprise a sensor applicator to deliver components of a medical device transdermally. Example medical devices that can be used with the disclosed and described technology include, and are not limited to, body wearable devices such as analyte sensors, pumps for the delivery of therapeutic drugs (insulin, chemotherapy drugs, etc.), and any other device as will be readily understood by those of skill in the art. Example medical device components that can be delivered transdermally with the embodiments disclosed and described herein include, and are not limited to, analyte sensing elements, drug delivery cannulas (microcatheters) or other delivery lumens for infusion pumps to deliver, for example, insulin and other therapeutic agents/treatments to a patient, etc. Additional items can be delivered with the embodiments disclosed herein including, and not limited to, drug eluting implants. For analyte sensors, analytes that can be measured using the embodiments of the invention disclosed and described herein include, and are not limited to, glucose, galactose, fructose, lactate, peroxide, cholesterol, amino acids, alcohol, lactic acid, and mixtures of the foregoing.

Analyte sensors, and components thereof, that may be used with the embodiments of the disclosed and described technology include, and are not limited to, those described in the following commonly assigned U.S. Pat. No. 11,553,879, entitled “SYSTEMS AND METHODS FOR CONTINUOUS HEALTH MONITORING USING AN OPTO-ENZYMATIC ANALYTE SENSOR,” filed Feb. 21, 2018, which is hereby incorporated herein by reference in its entireties for all purposes.

The sensor applicator may be an analyte sensor applicator for transdermally deploy an insertable portion of an analyte sensor in a user and for externally applying a wearable analyte monitoring device on the user's skin.

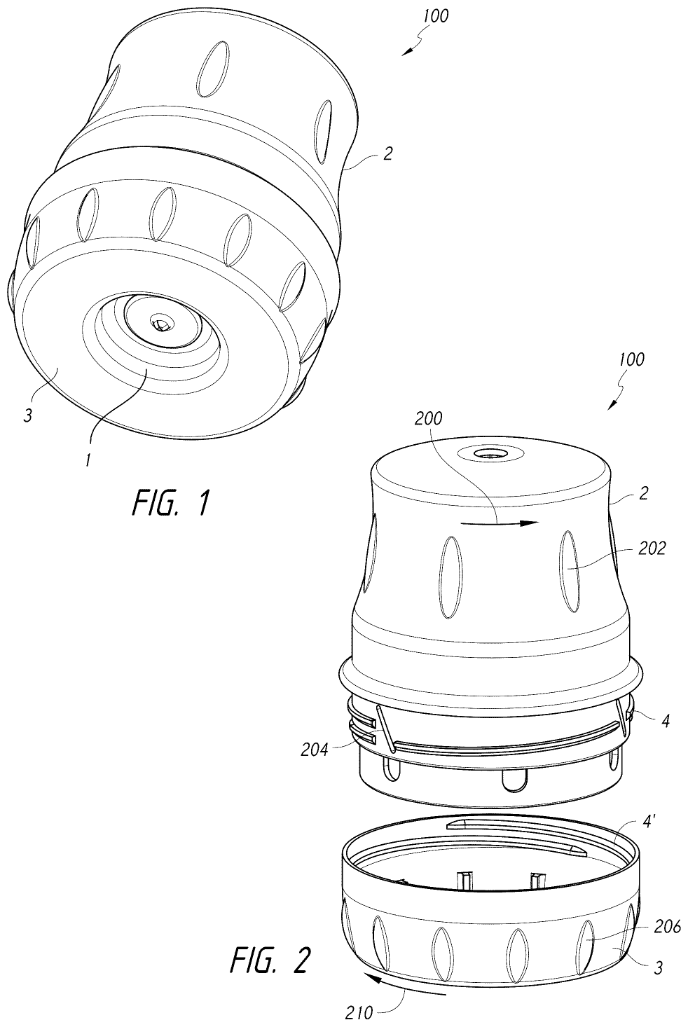

FIG. 1 depict an applicator 100 according to some embodiments. The applicator 100 may be an analyte sensor applicator. The analyte sensor applicator 100 may comprise a housing top 2 and housing bottom 3 that may be secured together. The housing bottom 2 may comprise an opening 1 at the bottom of the housing bottom 3. FIG. 2 illustrates a perspective view of the housing top 2 and the housing bottom 3. The housing top 2 may include one or more housing top features 202 to facilitate the opening of the applicator 100 and/or the holding of the applicator 100. The housing top 2 may include one or more perforations 204. The perforations 204 may be round, rectangular, square, oval, or any other shapes. The housing bottom 3 may include housing bottom features 206 to facilitate the opening of the applicator 100 and/or the holding of the applicator 100. The housing top 2 and housing bottom 3 may be secured together by mating threads 4, 4′. The applicator 100 may be opened by twisting, for example by rotating the housing top 2 in direction 200 and rotating the housing bottom 3 in direction 210. In alternative embodiments, the housing top 2 and housing bottom 3 may be secured together by bayonet-style connectors. In some embodiments, the housing top 2 and housing bottom 3 may be secured together by adhesive or tape.

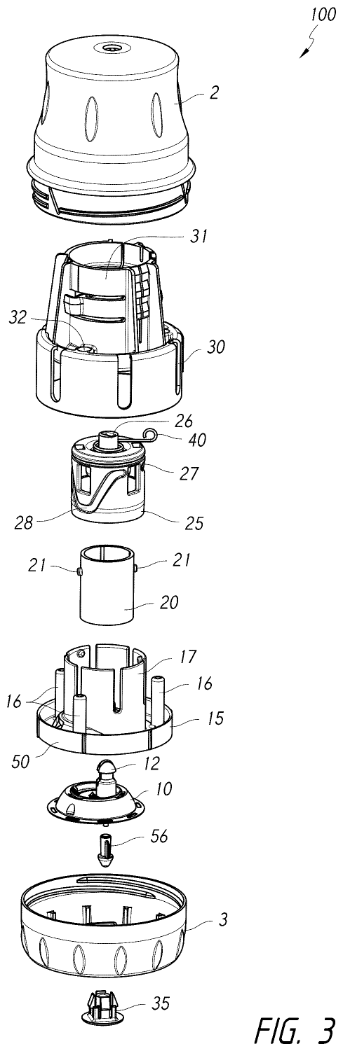

FIG. 3 illustrates an exploded view of the components of the analyte sensor applicator 100 according to some embodiments. As shown in FIG. 3, the applicator 100 may include the applicator housing top 2, an applicator frame 30, a spring 40, an applicator cam 25, an applicator retractor 20, an applicator piston 15, a chuck 12, a skin piercing device 6, a sterile cap 56, the housing bottom 3, and/or a sterile cap key 35. The applicator frame 30 may include one or more applicator frame openings 32 and/or an open center frame portion 31. The retractor 20 may include retractor one or more pins 21. The applicator piston 15 may include piston arms 16, a piston cylinder 17, and/or a piston base 50. The sterile cap 56 may be configured to cover the skin piercing-device 6 and/or an insertable portion of the sensor 5 (not shown).

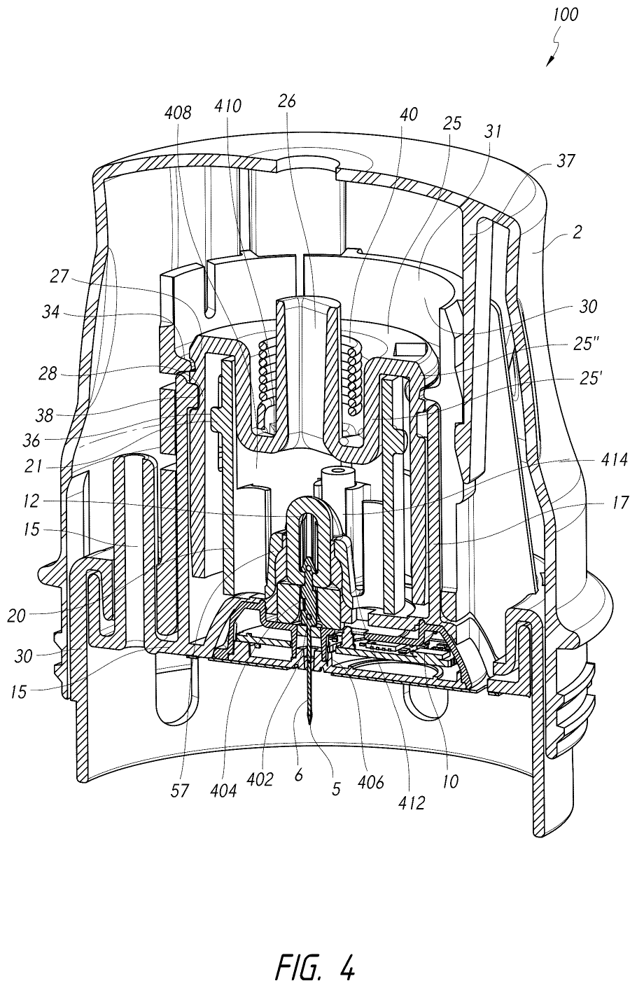

FIG. 4 illustrates a cross sectional view of the applicator 100 including the wearable analyte monitoring device 10 in an initial status after the removal of housing bottom 3 and prior to activation according to some embodiments. As illustrated in FIG. 4, the components of the applicator 100 may engage in a snap-fit arrangement. The housing top 2 may comprise mechanical connections to engage the applicator frame 30. For example, the tabs or cantilever 37 of the housing top 2 may engage with applicator frame 30. At the initial status, the cantilever 37 may be engaged with the outer surface of the open center frame portion 31 of the applicator frame 30 at a first position. Upon activation, such as the user pressing down the housing top 2, the cantilever 37 may be engaged with outer surface of the open center frame portion 31 of the applicator frame 30 at a second, distal position along the outer surface of the open center frame portion 31 to lock the housing top 2 at a distal position. The cantilever may be engaged with the applicator frame 30 via snap-fit, press-fit, or any other connection. In some embodiments, the housing top 2 may comprise mechanical connections to engage cam 25 and/or the piston 15. The cam 25 may comprise a circumferential groove 27 adjacent the top of the cam 25 that engage with one or more internal tabs 34 within the applicator frame 30.

With continued reference to FIG. 4, the applicator frame openings 32 may be configured to receive piston arms 16. The open center frame portion 31 of the applicator frame 30 may be configured to receive the piston cylinder 17 of the applicator piston 15 for alignment of the applicator piston 15 within the applicator frame 30. The cam may include an internal wall 25′ and an external wall 25″. The retractor 20 may be aligned between the internal 25′ and external 25″ cam walls. The cam 25 and retractor 20 may be disposed within the piston cylinder 17. In some embodiments, a cam assembly may comprise the cam 25, retractor 20, and/or applicator piston 15.

With continued reference to FIG. 4, the retractor 20 may include a receiving port 57 within, or on the bottom of, the retractor 20. The retractor 20 may include one or more slides 412. The skin piercing device 6 may be connected to the retractor 20 by the chuck 12. The skin piercing device 6 may include a proximal end 402 for engaging with the chuck 12. The chuck 12 may engage with the receiving port 57 on the bottom of the retractor 20. The connection between the chuck 12 and the receiving port 57 may be a snap-fit, press-fit, and/or adhesive engagement. The chuck 12 and the proximal end 402 of the skin piercing device 6 may be connected via a snap-fitted, welded, or glued connection. The skin-piercing device 6 may engage with an insertable portion 5 of a sensor.

With continued reference to FIG. 4, the wearable analyte monitoring device 10 may be under the piston base 50 and may be attached to the piston base 50. The contour of the concave piston base 50 may match the contour of a top cover of the wearable analyte monitoring device 10. The piston base 50 may include a piston base opening 404. The piston may include one or more inner piston arms 414 extending upwards from the piston base 50. The inner piston arms 414 may be configured to be received and slide inside of the piston slides 412. The wearable analyte monitoring device 10 may include a wearable device opening 406. The skin piercing device 6 may move through the piston base opening 404 and the wearable device opening 406.

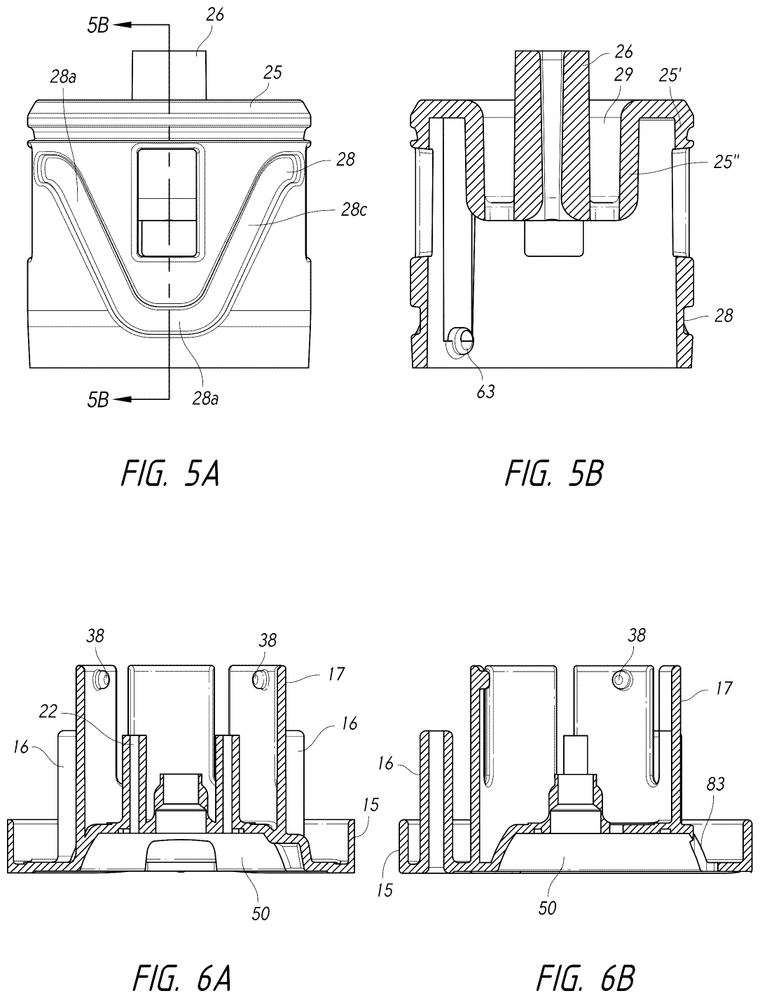

FIG. 5A illustrates a perspective view of the cam 25 according to some embodiments. FIG. 5B illustrates a cross section view of the cam 25 in FIG. 5A. As illustrated in FIGS. 5A-5B, the cam 25 can have an internal cam wall 25′ and external cam wall 25″. The external cam wall 25″ has an external surface 502 and an internal surface 504. One or both of the internal surface 504 and the external surface 502 can include one or more cam paths or grooves, for example, the internal surface 504 can include one or more internal cam paths or grooves 70 (not shown) and the external surface 502 may include an external cam path or groove 28. A cam path may be a sliding track rising from the surface.

As shown in FIG. 5A, the cam 25 can include an external groove 28 to guide the applicator piston 15 (see FIGS. 6A and 6B). The applicator piston 15 drives the wearable analyte monitoring device 10. The external groove 28 can include a first end and a second end. The external groove 28 can include a first section 28a, a second section 28b, and/or a third section 28c. The first section 28a can extend from the first end of the groove 28, the third section 28c can extend from the second end of the groove 28, and the second section 28b can extend therebetween. The first section 28a may be sloped from an upper portion of the cam 25 toward a lower portion of the cam 25. The second section 28a extends along a lower portion of the cam 25 and is oriented relatively horizontal compared to a longitudinal axis of the cam 25. The third section 28c extends from the lower portion of the cam 25 to the upper portion of the cam 25. The third section 28c may be oriented at the same angle as the first section 28b relative to the longitudinal axis of the cam 25.

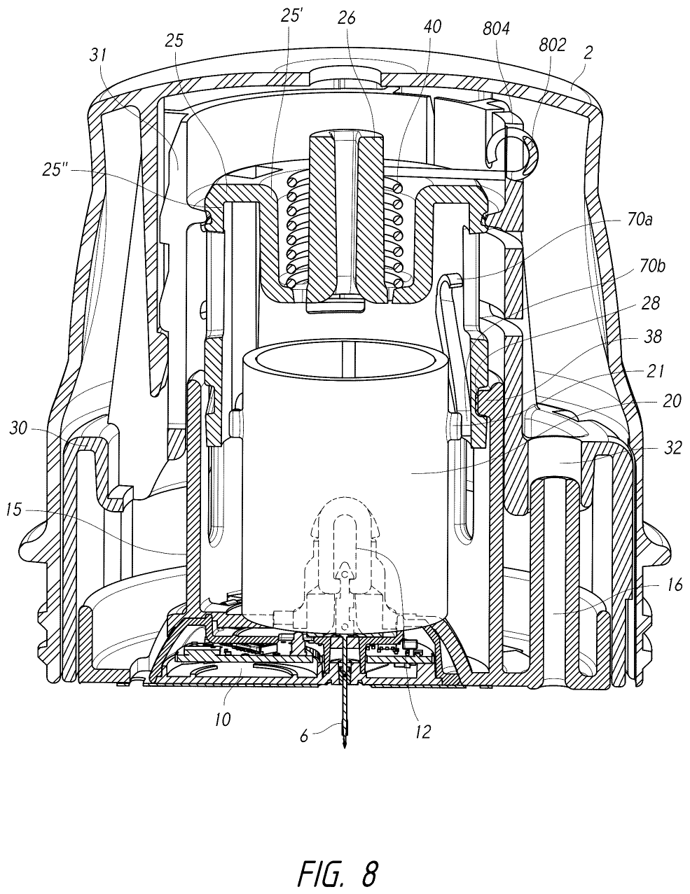

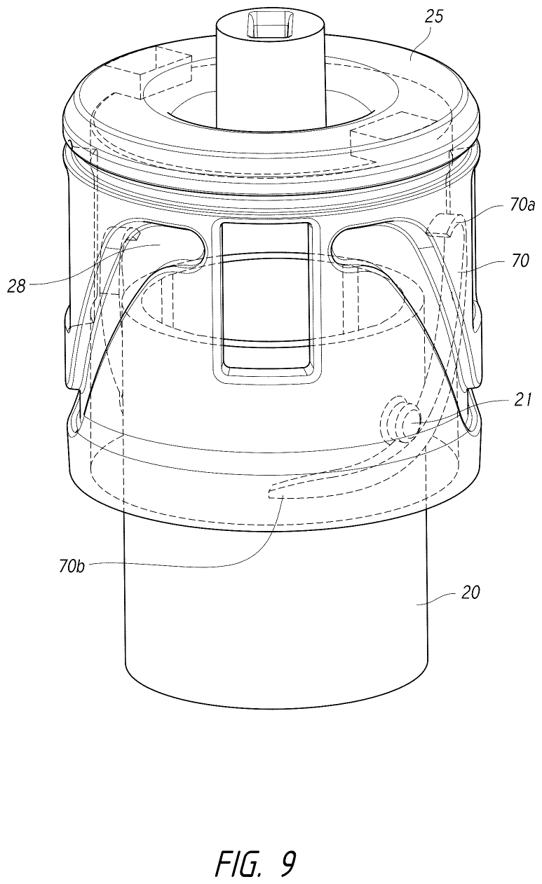

The internal cam path or groove 70 can be best seen in FIGS. 8 and 9. The internal cam path or groove 70 guides the retractor 20. The internal cam path or groove 70 can include a first end 70a and a second end 70b. The first end 70a can be positioned toward an upper portion of the cam 25, while the second end 70b can be positioned toward a lower portion of the cam 25.

FIGS. 6A and 6B illustrate different cross-section views of piston 15 according to some embodiments. The cam 25 is configured to align within the applicator piston 15. The applicator piston 15 slidably engages with external grooves 28 of the cam 25 via piston pins 38. As illustrated in FIG. 9, the retractor 20 is configured to align within the cam 25 and has external retractor pins 21 to slidably engage with internal cam grooves 36.

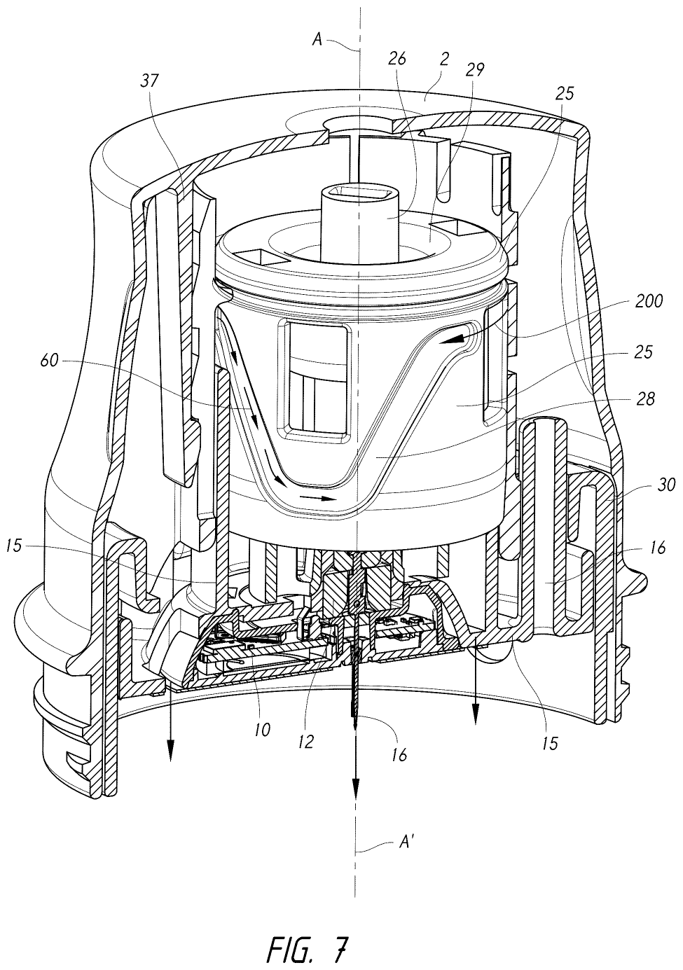

As a downward force is applied to the cam 25, the cam 25 may rotate. For example, as explained in more detail below, the activation of a loaded spring may cause the rotation of the cam 25. The rotation of the cam 25 may cause the applicator piston 15 to travel relative to the cam 25. As the applicator piston 15 travels along the first section 28a, the piston 15 moves towards a distal position to drive the wearable analyte monitoring device 10 toward the skin (see FIG. 7). In addition, the rotation of the cam 25 may cause the retractor 20 to travel relative to the cam 25, for example from the first end 70a to the second end 70b, to drive the skin-piercing device 6 through the skin. When the housing top 2 is pushed to the skin surface, the skin-piercing device 6 is deployed through the skin. As the applicator piston 15 travels along the second section 28b, the piston stays at the distal position. When the applicator piston 15 travels along the second section 28b, the skin piercing device 6 may further penetrate the skin tissue with the insertion momentum. In addition, second section 28b may provide a buffer between the insertion and retraction of the skin piercing device.

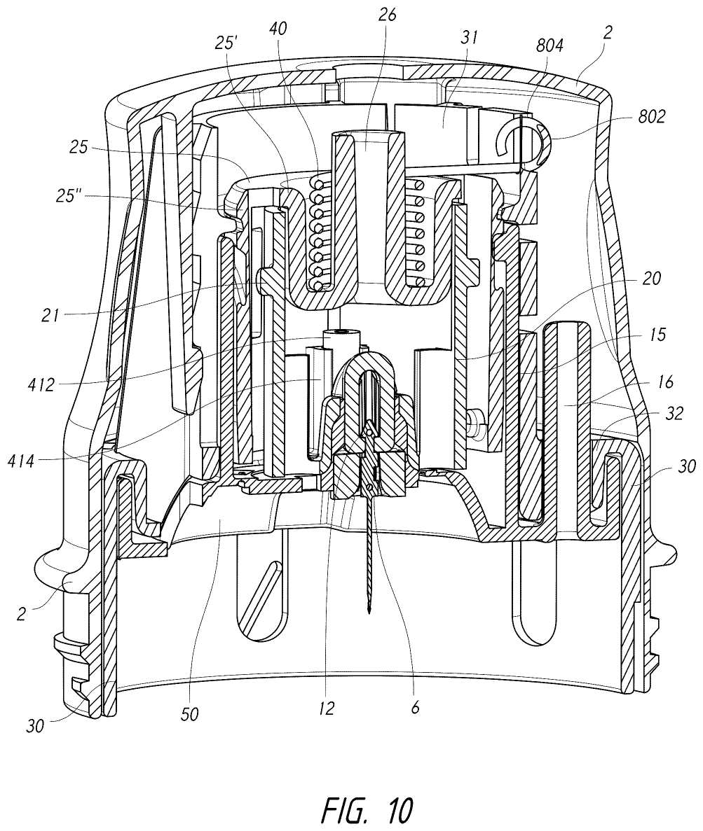

As explained in more detail below, a spring 40 may drive the cam continue to rotate such that the applicator piston 15 travels through the third section 28c of the groove 28. The piston 15 may retract when it travels through the third section 28c of the groove 28. This retraction step may be automatic in that the user does not need to release the housing top 2 or any other mechanism to retract the piston 15 and/or the skin piercing device 6. As shown in FIG. 10, as the skin-piercing device 6 is retracted, the housing top 2 remains in a distal position against the skin-surface. As the applicator piston 15 is moved through the third section 28c, the retractor 20 is forced to travel back from the second end 70b of the cam path or groove 70 toward the first end 70a of the cam path or groove 70 to retract the skin piercing device 6. The housing top 2 may be locked in this distal position. Features of the drive mechanism are described in more detail below.

With reference back to FIG. 4, a spring 40, such as a torsion, tension or compression spring, may be positioned between the cam 25 and the housing top 2 under a load. The spring 40 may be released upon pushing down the housing top 2. In some embodiments, the spring 40 may be positioned in the way configured to provide drive for the cam 25. In some embodiments, the spring 40 may be positioned in contact with the cam 25. The spring 40 may be positioned in a spring cavity 29 around the cam shaft 26. The spring 40 may comprise a first end that engages with the cam 25, for example, by wrapping around a hole at the bottom of the spring cavity 29 of the cam 25. A second end 802 of the spring 40 may engage with a notch 804 on an upper portion of the open center frame portion 31 of the applicator frame 30 (see FIG. 8). An uncompressed spring 40 may be placed under load by rotating the cam shaft 26 to wind the spring 40. The applicator 100 may be activated by pressing on the housing top 2, disengaging mechanical connections that lock the cam 25 to prevent rotation. Disruption of the connection between the applicator frame 30 and the housing top 2 to unlock the cam 25 and activate movement of component parts may be by a displaced cantilevered pawl, a pivoting release, twisting release or rotating release. For example, where the housing top 2 engages with slots on the applicator frame 30, for example, via a cantilevered pawl, the connection may be broken where the pawl is pulled back under tension by pushing, twisting or rotating motion of the housing top, releasing the cam 25.

With reference to FIGS. 6A and 6B, in some embodiments, the piston 15 comprises a concave piston base 50 that conforms with the geometry of the outwardly facing surface of the wearable device 10. As illustrated in FIG. 4, prior to activation, the wearable device 10 is contained within a concave portion of the piston base 50 within the housing top 2. Prior to activation of the applicator 100, the wearable analyte monitoring device 10 may be held within piston base 50 by mechanical means, such as a low stick adhesive located between the wearable device and the piston base.

With reference to FIG. 3, the wearable device 10 may be retained within the housing by a sterile cap key 35 located in the housing bottom 3. The sterile cap key 35 aligns with one or more components on the bottom of the wearable device 10 until removal of the housing bottom 3. This is described in further detail below with respect to FIG. 37.

In an alternative embodiment, one or more springs may be provided within the housing having an arm that engages with the wearable device and a spring portion that is held in place under load by the retractor. Upon retraction by the retractor, the spring arms release the wearable device from the piston base. In some embodiments, a magnet may be placed within a retractor that magnetically engages with a magnetic component within the wearable device, such as a battery. The magnetic connection that secures the wearable device in place within the house may be broken upon retraction of the retractor releasing the wearable device from the housing top 2.

As illustrated in FIG. 7, a rotational movement of the cam 25 may be converted to linear movement of the piston 15. As the piston arms 16 may be configured to be received by the applicator frame openings 32, the piston 15 may not rotate relative to the applicator frame 30 and may only move vertically parallel to the axis AA′. After the releasing of the cam 25, the cam 25 may rotate in the direction 200. As the cam 25 rotates, piston pins 38 follow an external cam groove 28 (directionally depicted by arrows 60). In some embodiments, the external cam groove 28 may have a U or V-shaped groove (as described above with respect to FIG. 5A) that goes from the top of the cam to the bottom of the cam and then goes from the bottom of the cam to the top of the cam. In some embodiments, the external cam groove 28 may have a L-shaped that goes from the top of the cam to the bottom of the cam and then remains parallel to the bottom edge of the cam 25. In other words, the groove 28 may only include a first section 28a and a second section 28b, without a third section 28c. The second section 28b may extend to the end of the groove 28. In some embodiments, there is only one groove on the external surface of the cam. In some embodiments, there are multiple grooves on the external surface of the cam 25.

With continued reference to FIG. 7, the piston 15 moves towards a distal position in a direction parallel with an axis of insertion (line A-A′) to drive the wearable device 10 toward the skin. The cam 25 continues to rotate after the piston 15 reaches the distal position and the piston pins 38 may continue to follow the groove 28. In some embodiments, the piston moves upward from the distal position if the groove is a U- or V-shaped groove. In some embodiments, the piston stays at the distal position if the groove is a L-shaped groove. In some embodiments, a catch or stop may be present on the piston rim to hold the piston to the frame and lock the piston in place in the distal position.

As illustrated in FIG. 4, the inner piston arm 414 may be configured to slide inside of the retractor slide 412. The retractor slides 412 may be parallel to the axis AA′ as illustrated in FIG. 7. As the piston 15 may only move in a direction parallel to the axis AA′ as discussed above, thus the retractor slides 412 may only move in a direction parallel tot eh axis AA′. As depicted in FIGS. 8 and 9, the rotational movement of the cam 25 may be converted to linear movement of the retractor 20. The external retractor pins 21 may engage cam path or groove 70 on the internal cam surface. In some embodiments, the cam path or groove 70 may be a track raising from the internal surface of external wall 25″ of the cam 25. In some embodiments, the cam path or groove 70 may be a groove. The internal cam path or groove 70 may extend from a first end 70a adjacent the top of the cam 25 to the second end 70b adjacent the bottom of the cam 25. With the releasing of cam 25, the retractor 20 moves towards the distal position in a direction parallel to the axis of the applicator during insertion. As illustrated in FIG. 7, during insertion, the piston 15 may move along the axis AA′ downwardly (direction of arrow 700) to drive skin-piercing device 6 into the user's skin. The insertable portion 5 of the analyte sensor attached to the skin-piercing device 6 may be inserted into the user's skin with the skin-piercing device.

FIG. 8 illustrates the applicator at an insertion position. As illustrated in FIG. 8, the retractor 20 may reach a distal position for inserting the skin-piercing device 6 with the insertable portion 5 of the sensor into the user. The retractor 20 may reach the distal position when the external retractor pins 21 reaches the second end 70b the cam path or groove 70. The piston 15 may reach a distal position for placing the wearable device 10 on the user's skin when the piston pins 38 reach the second section 28b of the external groove 28 on the external surface of the cam 25.

The slope and shape or pattern of external and/or internal grooves on the cam may be varied to increase or decrease the speed or force at which the piston or retractor move up or down, or to select the number of times either part moves up or down, or whether or not either part moves up or down. In one embodiment, the piston and the retractor may be designed to move independently of the other.

FIG. 10 illustrates the cross section view of the applicator 100 after insertion of the sensor into the user and the placement of the wearable device 10 on the user's skin according to some embodiments. As illustrated in FIG. 10, after insertion, the piston 15 may move upwards as the external piston pins 38 move into the third section 28 of the external groove 28 on the outer surface of the cam 25. With the upward movement of the piston 15, the retractor 20 may move upward with the piston as the bottom of the retractor 20 may be in contact with the outer surface of the piston base 50. Both the piston 15 and skin piercing device 6 may be retracted into the housing top 2. In some embodiments, after insertion, the piston 15 may locks into the distal position to secure the wearable analyte monitoring device 10 against the skin of a user to prevent sliding or twisting. For example, the groove 28 may be an L-shaped groove such that the piston 15 may not move upwards. In another example, a stop may lock the piston 15 at the distal position. In some embodiments, the retractor 20 may retract independently from the piston 15. For example, the internal cam path or groove 28 may further comprise a third end 70c that extend from the second end 70b toward the top of the cam 25, such that the retractor 20 may move upwards as the retractor pins 21 slide in the cam path or groove 28 with the rotation of the cam 25. After the independent retraction of the retractor, the skin piercing device 6 may be retracted through the in the piston base opening 404 and secured within the housing top 2 above the piston base 50 to prevent the exposure of the skin-piercing device to the suer.

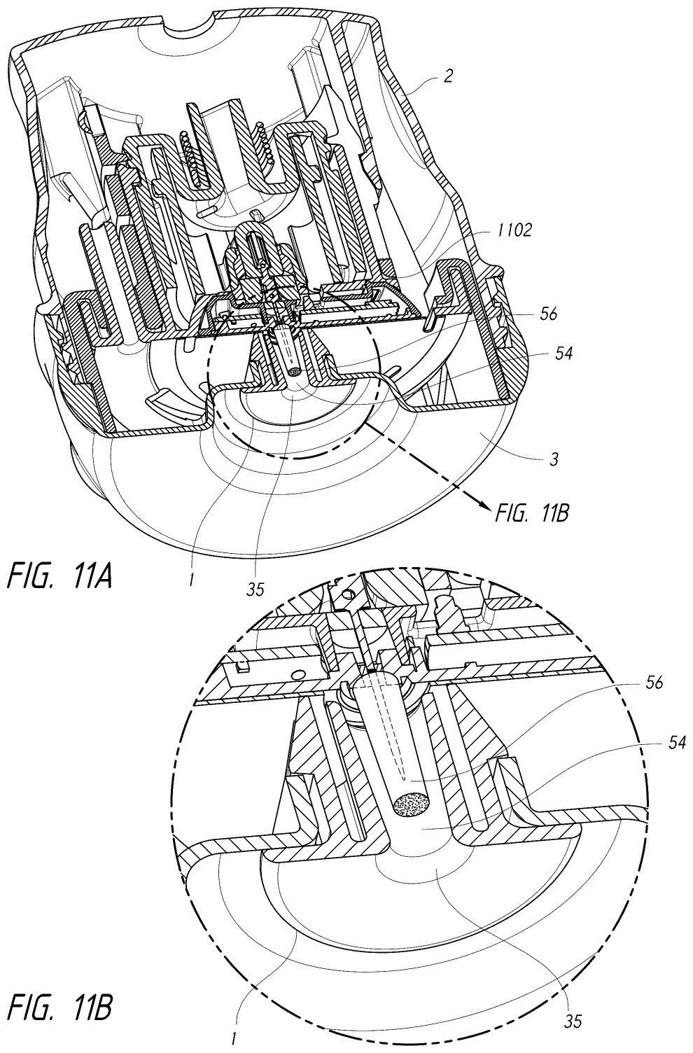

FIGS. 11A and 11B illustrate the cross section view of the final product of the applicator 100 before use. As illustrated in FIGS. 11A and 11B, the wearable device 10 is housed inside of the piston base 50. The housing top 2 and housing bottom 3 are secured together. The skin-piercing device 6 is covered by a sterile cap 56. The sterile cap key 35 is inserted into the bottom housing through the opening 1 of the bottom housing 2. In some embodiments, the sterile cap key 35 may be fixed in the opening of the bottom housing via snap-fit. In some embodiments, the sterile cap may be in touch with the bottom of the wearable device 10 and hold the wearable device 10 against the piston base 50. Sterilization features are described in more detail below.

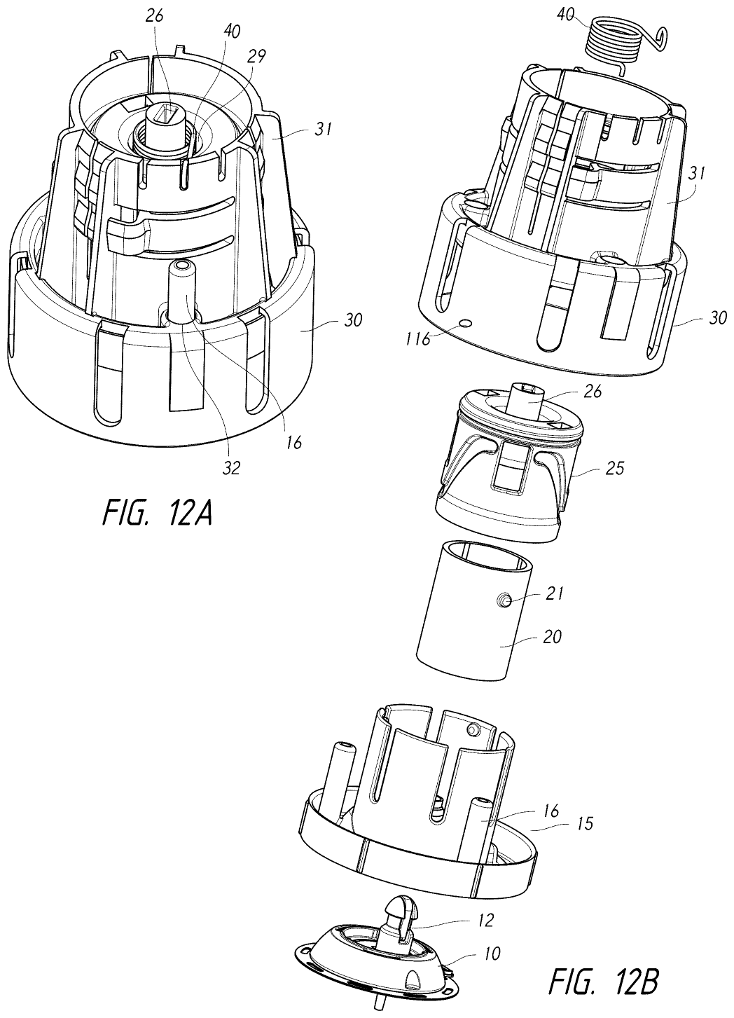

FIG. 12A illustrates a perspective view of the assembled applicator 100 inside of the applicator housing. FIG. 12B illustrates an exploded view of different components of assembled applicator 100 in FIG. 12A. As illustrated in FIG. 12A, the spring 4 is positioned in the spring cavity 29 around the cam shaft 26 comprises a first end that engages with the cam 25. The applicator frame 30 comprises openings 32 configured to receive piston arms 16 and an open center frame portion 31 configured to receive the piston cylinder 17 for alignment of the piston 15 within the applicator frame 30. As the piston arms 16 is placed inside of the openings 32, the piston cannot rotate during the rotation of the housing bottom, such that the rotation of the piston is prevented.

In alternative embodiments, the shape of the different components of the applicator 100 discussed may be different from the shapes illustrated in the figures, as long as the different components of the applicator can be assembled together to perform the function of the applicator 100.

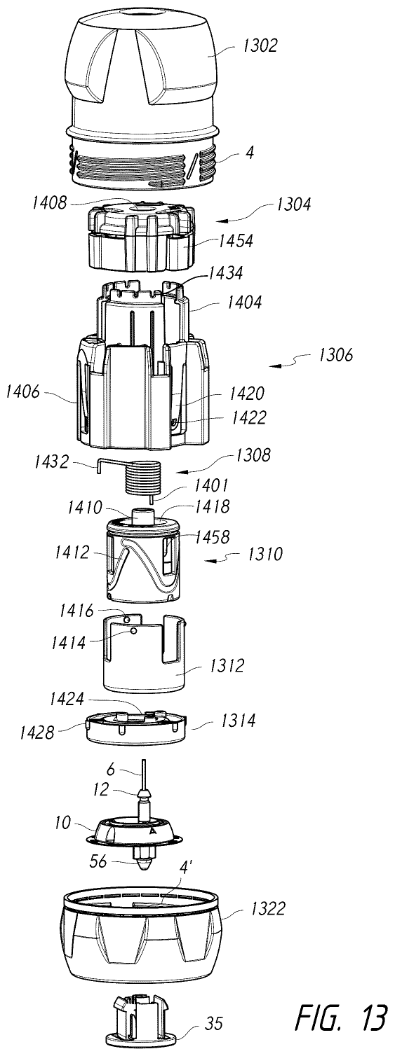

An alternative embodiment of an applicator 1300 is illustrated in FIG. 13. The applicator 1300 can include any of the features described above with respect to applicator 100. But unlike applicator 100, the applicator 1300 only includes a single thrust barrel 1312 for driving both the wearable device 10 and the skin piercing element 6. In this design, the drive cam 1310 only includes cam path(s) on the internal or external wall of the drive cam 1310.

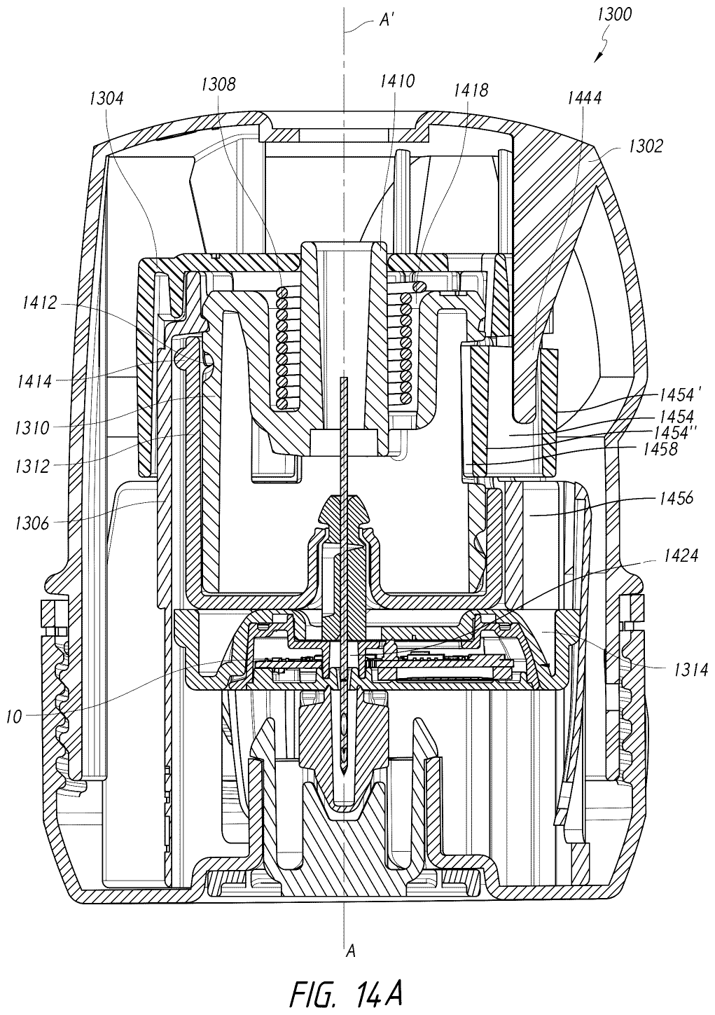

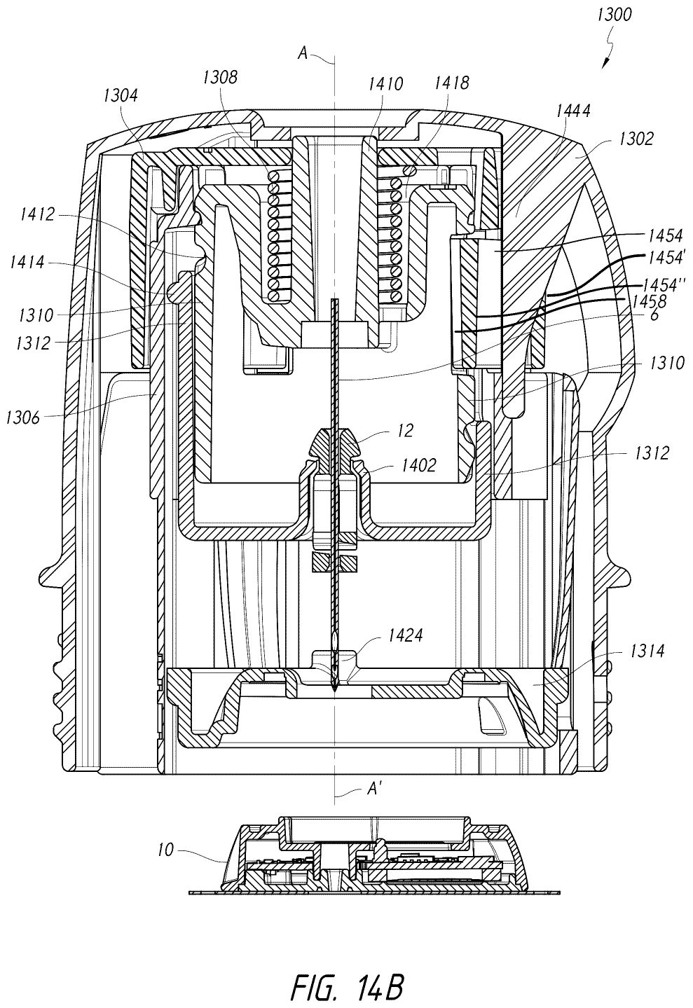

FIG. 13 illustrates an exploded view of components of another embodiment of applicator 1300 for transdermally inserting an analyte sensor tip in a user and for externally applying a wearable analyte monitoring device 10. FIG. 14A illustrates a cross sectional view of the applicator 1300 including a wearable analyte monitoring device 10 before activation. FIG. 14B illustrates a cross sectional view of the applicator 1300 including a wearable analyte monitoring device 10 after activation. As illustrated in FIGS. 13 and 14A-14B, the applicator 1300 may comprise a housing top 1302, a cam lock 1304, an applicator frame 1306, a spring 1308, a drive cam 1310, a thrust barrel 1312, a piston 1314, a skin-piercing device 6, a chuck 12, a sterile cap 56, a housing bottom 1322, and/or a sterile cap key 35. Any of these features may be removed from the applicator 1300 and should not be considered essential. Additionally, any of these features may be combined with features of the applicator 100.

The housing top 1302 and housing bottom 1322 may be secured together, for example, by mating threads 4, 4′. In alternative embodiments, the housing top 2 and housing bottom 3 may be secured together by bayonet-style connectors. In some embodiments, the housing top 2 and housing bottom 3 may be secured together by adhesive or tape. The sterile cap key 35 may be inserted through a hole on the bottom of the housing bottom 1322.

With continued reference to FIG. 13, the skin-piercing device 6 may connect to the thrust barrel 1312 through a chuck 12 that engages with a receiving component 1402 within or on the bottom of the thrust barrel 1312. The applicator chuck 12 and a top portion of a skin-piercing device 6 may be connected via a snap-fitted, welded, or glued connection. In some embodiments, the connection between the chuck 12 and the thrust barrel 1312 may be via snap-fit, press-fit, or adhesive engagement, with the receiving component 1402 within, or on the bottom of, the thrust barrel 1312. As illustrated in FIGS. 14A-14B, the chuck 12 is pushed into the thrust barrel 1312 through the opening of the receiving component 1402 and the edge of opening of the receiving component 1402 may be stuck in the concave neck of the chuck 12.

The design of the receiving port 57, the chuck 12 and the skin piercing device 6 depicted in FIG. 4 may also be configured to be used together with this design of applicator. The design of the receiving component 1402, the chuck 12 and the skin-piercing device 6 illustrated in FIGS. 13 and 14A-14B may also be used with the first design.

The components of the applicator may engage in a snap-fit arrangement. As illustrated in FIGS. 13 and 14A-14B, the applicator frame 1306 may comprises an upper portion 1404 and a lower portion 1406. The upper portion 1404 may be configured to match the cam lock 1304, such that the cam lock 1304 can be fit onto the upper portion 1404 of the applicator frame 1306. The upper portion 1404 may comprise one more notch 1434. In some embodiments, the upper portion 1404 of the applicator frame 1306 may comprise alignment features that indicate the correct placement of the cam lock 1304. The cam lock 1304 may comprise a center hole 1408 to receive the cam shaft 1410 of the drive cam 1310. The cam lock 1304 may include one or more receiving ports 1454.

The cam lock 1304 can maintain spring 1308 in a loaded configuration prior to use. As shown in FIG. 13, the spring 1308 may include a first end 1401 and a second end 1432. The first end 1401 interface with the cam cavity 1418. The second end 1432 may be configured to interface with the applicator frame 1306. The second end 1432 of the spring 1308 may be placed in one of the notches 1434 on the upper portion 1404 of the applicator frame 1306 during assembling the applicator 1300. Different notches may be associated with different strength of the spring after assembling and winding the spring 1308. The applicator frame 1306 may maintain the position of the spring 1308 until a downward force is applied to the housing top 1302 to release the cam lock 1304. As illustrated in FIG. 14A, the applicator housing top 1302 may include one or more cantilevered pawls 1444. The cantilevered pawl 1444 may be configured to be inserted into a receiving port 1454 of the cam lock 1304. The receiving port 1454 of the cam lock 1304 may include an internal wall 1454″ and an external wall 1454′. The internal wall 1454″ of the receiving port 1454 may be configured to fit in the through hole 1458 of the drive cam 1310 before activation of the applicator 1300. As illustrated in FIG. 14B, after activation, for example, by pushing the housing top 1302, the cantilevered pawl 1444 is pushed down with the housing top 1302. The cantilevered pawl 1444 is pushed further into the receiving port 1454 of the cam lock 1304. As the cantilevered pawl 1444 has a tapered cross section, with the further insertion of the cantilevered pawl 1444 into the receiving port 1454 of the cam lock 1304, the receiving port 1454 of the cam lock 1304 is pushed outwards such that the internal wall 1454″ of the receiving port is pulled out of the through-hole 1458 of the drive cam 1310. Thus, the drive cam 1310 may be released to rotate after activation of the applicator 1300.

With continued reference to FIG. 13, the applicator frame 1306 may comprise one or more deflectable portions 1420 extending from the top of the lower portion 1406 of the applicator frame 1306. The deflectable portion 1420 may comprise a free end which is not connected to the applicator frame 1306. In some embodiments, the deflectable portion 1420 may slightly curve towards the inside of the applicator frame 1306. The deflectable portion 1420 may include a locking hole 1422 adjacent the free end of the deflectable portion 1420. In some embodiments, the deflectable portion 1420 may be flexible. In some embodiments, the deflectable portion 1420 may be configured to be pushed outwardly. The deflectable portion 1420 may include a deflectable portion slot on the internal surface of the deflectable portion extending from the top of the lower portion 1406 to the locking hole 1422.

With continued reference to FIGS. 13 and 14A-14B, the cam 1310 may comprise an external groove 1412 on the external surface of the cam 1310. The external groove 1412 can include any of the features of the cam path or groove described above with respect to FIG. 5A. The drive cam 1310 can drive the thrust barrel 1312 in a similar manner to the applicator piston 15 described above. In some embodiments, the drive cam 1310 comprises a plurality of grooves on the external surface of the drive cam 1310. For example, each groove can drive a corresponding pin in the thrust barrel 1312. In some embodiments, the plurality of grooves are identical. In some embodiments, the plurality of grooves are not identical. In some embodiments, the drive cam 1310 does not comprise grooves on the internal surface of the drive cam 1310. In some embodiments, the drive cam 1310 comprises one or more grooves on the internal surface of the drive cam 1310 and does not comprise any groove on the external surface of the drive cam 1310. The thrust barrel 1312 may comprise thrust barrel pins 1416 that fit in the grooves 1412. The diameter of the drive cam 1310 may be configured to fit in the thrust barrel 1312 such that the thrust barrel pins 1416 can move smoothly along the grooves 1412 without falling out of the grooves 1412. In some embodiments, the thrust barrel 1312 may comprise one or more alignment features 1414 which may match with vertical slots parallel to the applicator axis AA′ on the internal surface of the applicator frame 1306, such that the thrust barrel 1312 may be placed in the applicator frame 1306 in the correct placement. During the movement of the thrust barrel 1312 inside of applicator frame 1306, the alignment features 1414 may confine the movement of the thrust barrel 1312 in a direction parallel to the axis AA′ of the applicator. Although the thrust barrel 1312 is depicted as having internal pins riding along an external cam path along the drive cam 1310, the components may be reversed such that external pins on a thrust barrel 1312 ride along an internal cam path of the drive cam 1310. The drive cam 1310 may include one or more through-holes 1458 on the cylindrical wall.

With continued reference to FIG. 13, the piston 1314 may comprise a piston opening 1424 for the skin-piercing device 6 to be inserted and retracted therethrough. The piston 1314 may comprise one or more piston locking features 1428 on the outer surface of the piston 1314. The piston locking features 1428 may lock the position of the piston when some of the piston locking features 1428 enter the locking holes 1422 adjacent the free end of the deflectable portions 1420 after sliding down along the slots of the deflectable portions 1420 of the applicator frame 1306. Some of the piston locking features 1428 may be fit in applicator frame slots on the internal surface of the lower portion 1406 of the applicator frame 1306. The applicator frame slots may be parallel to the applicator axis AA′ as shown in FIGS. 14A-14B.

With continued reference to FIG. 13, the spring 1308, such as a torsion, tension or compression spring 1308 is positioned between the drive cam 1310 and the cam lock 1304 under a load that releases upon pushing down on the housing top 1302. In some embodiments, the spring 1308 may be positioned in the way configured to provide drive for the cam 1310. In some embodiments, the spring 1308 may be positioned in contact with the cam 1310. In some embodiments, the spring 1308 is positioned in a spring cavity 1418 around the cam shaft 1410 comprises a first end 1401 that engages with the cam 1310, for example, by wrapping around a hole in the bottom of the cam 1310. A second end 1432 of the spring engages with a notch 1434 on an upper portion of the applicator frame. An uncompressed spring may be placed under load by rotating the cam shaft 1410 to wind the spring 1308. The applicator 1300 may be activated by pressing on the housing top 1302, releasing the cam lock 1304. Disruption of the connection between the cam lock 1304 and the housing top 1302 to unlock the cam 1310 and activate movement of component parts may be by a displaced cantilevered pawl, a pivoting release, twisting release or rotating release. Releasing the spring 1308 drives rotation of the drive cam 1310.

With reference to FIG. 13, the piston 1314 may conform with the geometry of the outwardly facing surface of the wearable device 10. Prior to activation, the wearable device 10 may be placed under the piston 1314 within the housing top 1302. Prior to activation of the device, the wearable analyte monitoring device 10 may be held under the piston 1314 by mechanical means, such as a low stick adhesive located between the wearable device and the piston. In some embodiments, a magnet may be placed within thrust barrel 1312 and/or piston 1314 that magnetically engages with a magnetic component within the wearable device 10, such as a battery. The magnetic connection that secures the wearable device in place within the house may be broken upon retraction of the thrust barrel 1312 releasing the wearable device from the housing top 1302. In an alternative embodiment, the wearable device may be retained under the piston by the sterile cap key 35 located in the housing bottom 1322. The sterile cap key 35 aligns with one or more components on the bottom of the wearable device until removal of the housing bottom 1322. In some embodiments, a magnet may be placed within thrust barrel 1312 and/or piston 1314 that magnetically engages with a magnetic component within the wearable device 10, such as a battery. The magnetic connection that secures the wearable device in place within the house may be broken upon retraction of the thrust barrel 1312 releasing the wearable device from the housing top 1302.

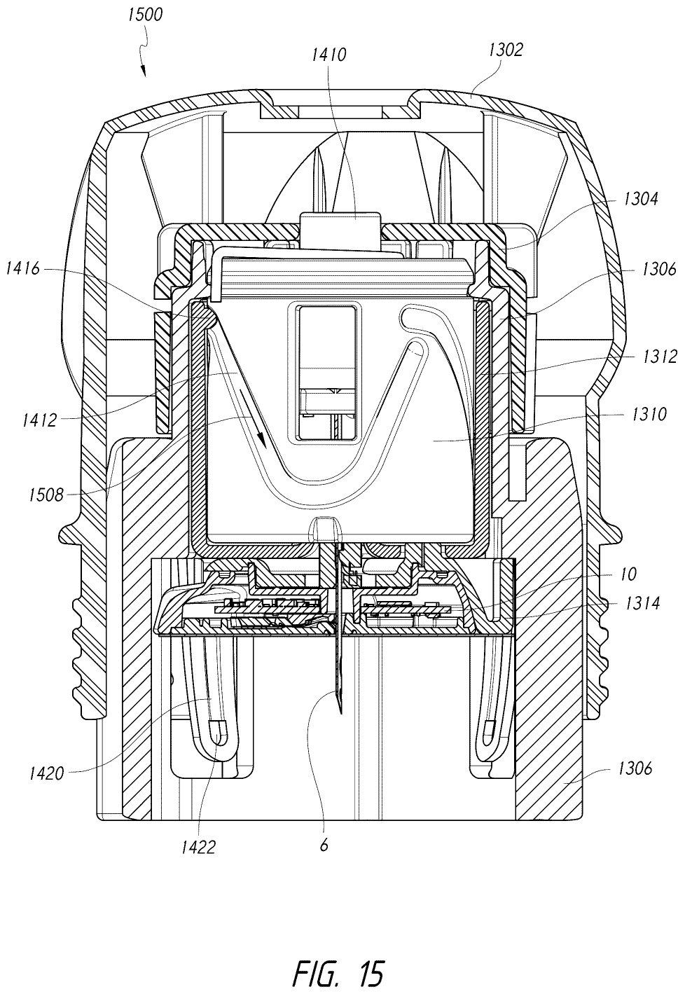

FIGS. 15-17 illustrate the movement of different components during insertion and retraction of the analyte sensor applicator 1300 according to some embodiments. A rotational movement of the drive cam 1310 may be converted to the linear movement of the thrust barrel 1312 and piston 1314. FIG. 15 illustrates an initial status before the activation of the applicator 1300. As illustrated in FIG. 15, the housing top 1302, the thrust barrel 1312, and/or the piston 1314 are at a first, proximal position. A user may press down the housing top 1302 to start the insertion of the skin-piercing device and the placement of the analyte monitoring device 10 on the user's skin. The applicator 1300 may be triggered by other means. After the housing top 1302 is pressed down, the cam lock 1304 may be released as described above, thereby releasing the spring 1308 and allowing the cam 1310 to rotate. As the cam 1310 rotates, the thrust barrel pins 1416 follow an external cam groove 1412 in the direction of arrow 1508. The thrust barrel 1312 can also include alignment features 1414 which can only move in the vertical slots on the internal surface of the upper portion 1404 of the applicator frame 1306, such that the thrust barrel 1312 is confined to move in a vertical direction parallel to the axis AA′ of the applicator 1300 (see FIGS. 14A-14B). As the thrust barrel 1312 may be connected to the skin-piercing device 6 through chuck 12, the skin-piercing device 6 is pushed to a distal position, such that the skin-piercing device 6 is inserted into the user. The movement of the thrust barrel 1312 may also push the piston 1314 to move distally parallel to the axis AA″ of the applicator 1300, such that the wearable device 10 is pushed against the skin of the user. When the thrust barrel pins 1416 reaches the lowest position of the groove 1412, the applicator 1300 reaches the distal position that the skin-piercing device 6 is inserted into the user's skin and the analyte monitoring device 10 is placed against the user's skin. FIG. 14B illustrates such an insertion position. At the insertion position, the piston 1314, the thrust barrel 1312 and/or the housing top 2 are at a second, distal position. At the distal position, the locking features 1428 on side surface of the piston 1314 may enter the matching locking holes 1422 at the free end of the deflectable portions 1420 of the applicator frame 1306 after sliding along the slots on the internal surface of the deflectable portion 1420, such that the piston 1314 is locked at the distal position.

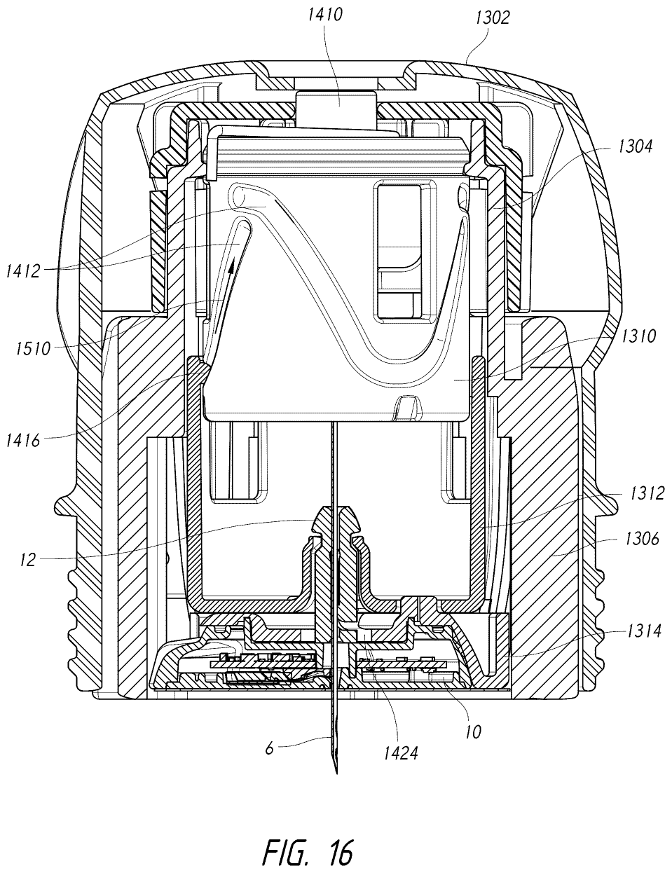

FIG. 16 illustrate the cross section view of the applicator 1300 during the retraction stage. As illustrated in FIG. 16, after the thrust barrel pins 1416 reaches the lowest position of the groove 1412, the drive cam 1310 may continue to rotate, such that the thrust barrel pins 1416 continue to follow the external cam groove 1412 in the direction of arrow 1510 and the thrust barrel 1312 retracts. This retraction step may be automatic from the continuous unwinding of spring 1308. The spring 1308 causes the thrust barrel 1312 to continue to travel relative to the drive cam 1310. As the thrust barrel retracts, it pulls up the chuck 12 and the skin-piercing device 6 through the piston opening 1424 of the piston 1314 while the piston stays at the distal position.

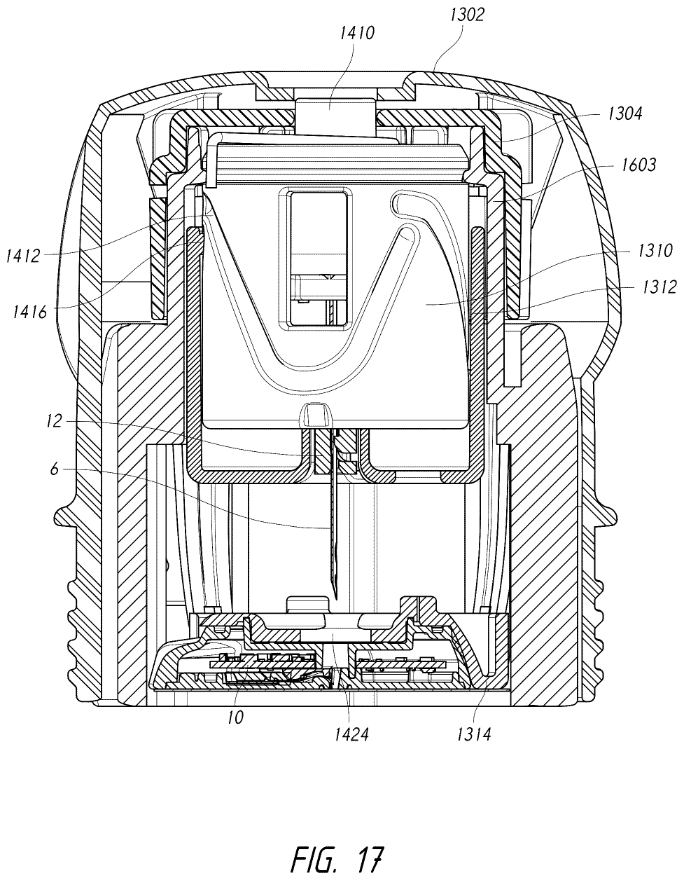

FIG. 17 illustrates a cross section view of the applicator 1300 after retraction of the skin-piercing device 6. As illustrated in FIG. 17, the skin-piercing device 6 has been retracted into the housing top 1302 above the piston 1314 while the piston 1314 stays at the distal position. The piston 1314 locked into the distal position secure the wearable analyte monitoring device 10 against the skin of a user. The piston locked into the distal position and/or the housing top 1302 being pressed down may stabilize the skin for insertion and retraction and may prevent sliding or twisting upon independent retraction of the skin-piercing device by the retractor. After insertion, the skin piercing device 6 is retracted through the piston opening 1424 through the piston 1314 and secured within the housing top 1302 above the piston base to prevent injury to the user.

In some embodiments, the slope, shape and pattern of drive cam groove at least partially determines the direction, force and speed of the movements of the moving components (e.g., thrust barrel, chuck, skin-piercing device, analyte monitoring device). In some embodiments, if the slope of the drive cam groove is smoother, the movement of the thrust barrel may be slower, and the force applied to the thrust barrel is smaller. In some embodiments, if the slope of the drive cam groove is steeper, the movement of the thrust barrel is faster and the force applied to the thrust barrel is larger. If the slope is too smooth, the skin-piercing device moves too slow, it may cause pain to a user. If the slope is too steep, the force applied to the analyte monitoring device 10 and the skin-piercing device 6 is too large and may also causes pain to a user.

In some embodiments, the force of the spring 1308 may impact the rotational speed of the drive cam 1310. In some embodiments, the increase of the force of the spring 1308 may decrease the rotational speed of the drive cam 1310, such that the speed of insertion and retraction may be slower. In some embodiments, the decrease of the force of the spring may increase the rotational speed of the drive cam, such that the speed of the insertion and retraction may be increased. As illustrated, the spring 1308 is generally oriented such that a longitudinal axis of the spring 1303 is aligned or parallel with a longitudinal axis of the applicator.

In alternative embodiments, the shape of the different components of the applicator 1300 discussed may be different from the shapes illustrated in the figures, as long as the different components of the applicator can be assembled together to perform the function of the applicator 1300.

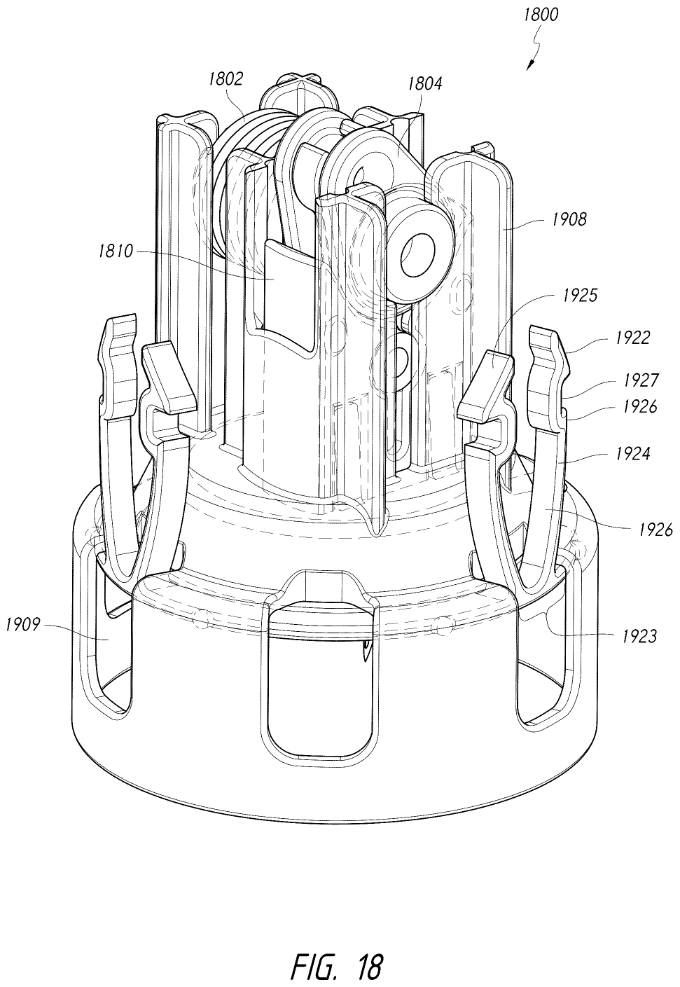

An alternative embodiment of an applicator 1800 is illustrated in FIG. 18. FIG. 18 illustrates a perspective view of the analyte sensor applicator 1800 absent a housing. The applicator 1800 may be housed in a suitable housing comprising a housing top and a housing bottom, similar to the designs discussed above.

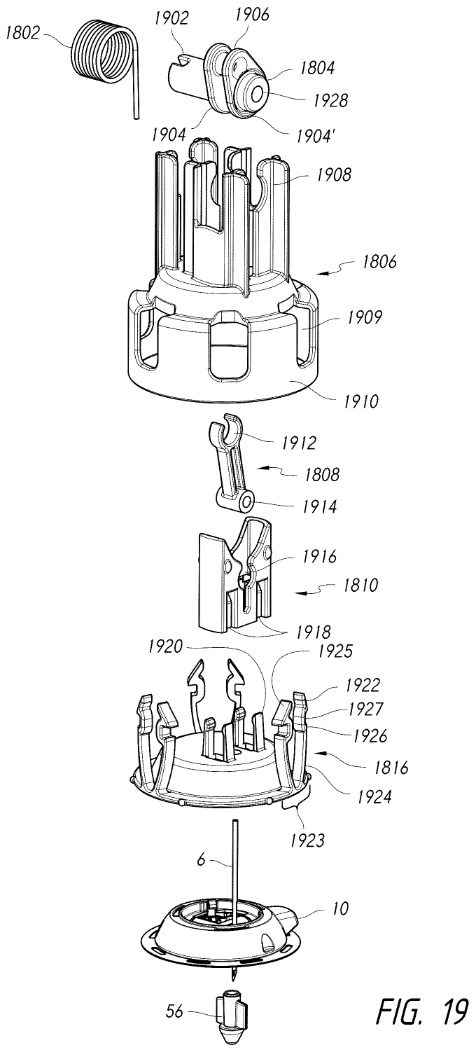

FIG. 19 illustrates an exploded view of the components of the analyte sensor applicator 1800 according to the embodiment in FIG. 18. As illustrated in FIG. 19, the analyte sensor applicator 1800 may comprise a spring 1802, a crankshaft 1804, an applicator frame 1806, a connecting rod 1808, a piston 1810, a skin piercing device 6, and/or a lancet shield 1816. A wearable analyte monitoring device 10 can be placed beneath the lancet shield 1816 when assembling the analyte applicator 1800. The crank mechanism described below may be responsible for driving the piston 1810, which may be coupled with the skin-piercing device 6. The piston 1810 may act on the lancet shield 1816 to drive the wearable device 10 toward the user's skin.

As illustrated in FIGS. 18 and 19, the applicator 1800 may include a crankshaft 1804 coupled with frame 1806. For example, the crankshaft 1804 may be received by the receiving end 1908 of the frame 1806.

The crankshaft 1804 may include one or more cams, for example two identical, parallel cams 1904, 1904′. As illustrated, the cams 1904, 1904′ can be eccentric. A rod portion 1902 may extend from one cam 1904. The rod portion 1902 may carry the spring 1802. A crankpin 1906 may extend between the two parallel cams 1904, 1904′. In some embodiments, the crankpin 1906 can positioned at an end of the cams 1904, 1904′ instead of between the cams. For example, the crankpin 1906 may be positioned at the end of the cams 1904, 1904′ further away from a rotating center. The end portion 1928 may extend from the other cam 1904′ for interfacing with the applicator frame 1806.

The connecting rod 1808 comprises a first end 1912 and a second end 1914. The first end 1912 may connect to the crankpin 1906 of the crankshaft 1804. In some embodiments, the first end 1912 may be connected to the crankpin 1906 with snap-fit, press-fit, welding, or any other connection methods. The second end 1914 of the connecting rod 1808 can be connected to the piston 1810. In some embodiments, the second end 1914 of the connecting rod 1808 may be pushed and snap-fit into the hole 1916 of the piston 1810. The piston 1810 can include a receiving end configured to be connected to the skin-piercing device 6 via a chuck (similar to the previous designs). The mechanism may be similar to those discussed in the previous embodiments.

The lancet shield 1816 may comprise one or more connecting features 1923 to connect the lancet shield 1816 to the applicator frame 1806. As illustrated, the connecting feature 1923 may include one or more branches 1926. Each branch 1926 of the connecting features 1923 may comprise a curved bottom portion 1924. Each branch 1926 of the connecting features 1923 may further comprise a snap fit structure 1922 at the end of each branch 1926. The snap fit structure 1922 can be a cantilever snap fit structure comprising a concave neck 1927 and a tilted portion 1925.

The lancet shield 1816 may be connected to the applicator frame 1806 by placing the connecting features 1923 into the one or more holes 1909 of the bottom portion 1910 of the applicator frame 1806. In some embodiments, the connecting features 1923 may be pulled through the holes 1909 by pinching the tilted portions 1925. The lancet shield 1816 may include connecting extrusions 1920 which may be fully engaged into the slots 1918 of the piston 1810.

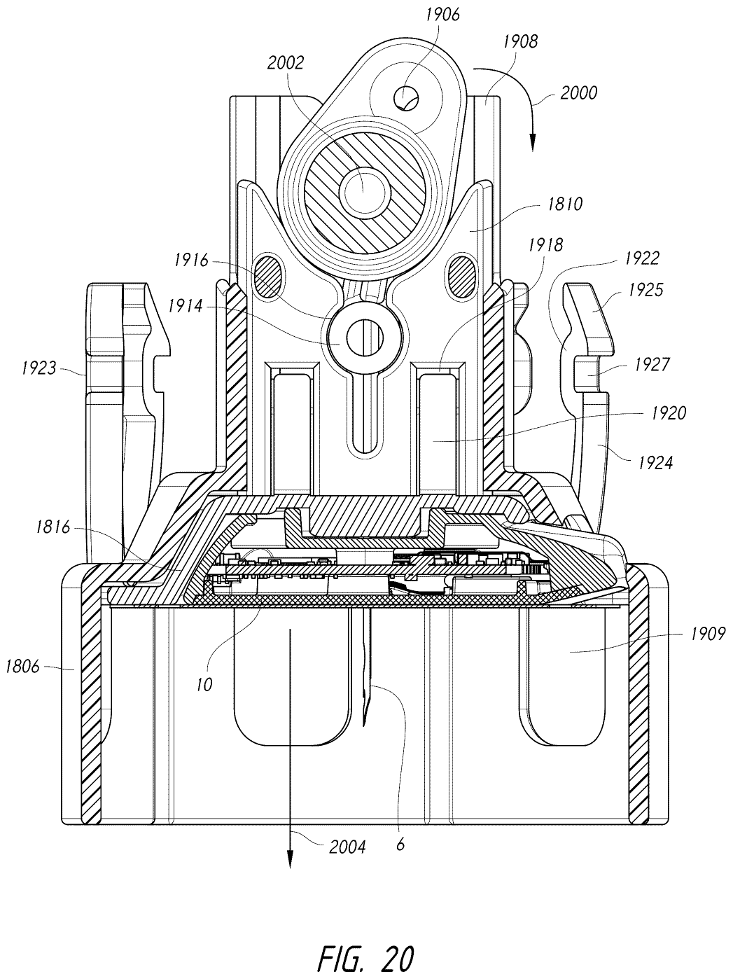

FIGS. 20-22 illustrate the cross section views of the applicator 1800 during the insertion and retraction of the skin-piercing device 6 and the placement of the wearable analyte monitoring device 10 against a user's skin. The different steps illustrated in FIGS. 20-22 can be the result of one full turn of the crankshaft 1804. Once the crankshaft 1804 begins rotation, the applicator 1800 can automatically and continuously move through each of the steps shown in FIGS. 20-22.

FIG. 20 illustrates the starting position before the user activates the applicator 1800. At the starting position, at least a portion or substantially the whole connecting feature 1923 is positioned above the holes 1909 in the applicator frame. The crankpin 1906 can be positioned above the rotating center 2002 of the crankshaft 1804.

An application of force to the housing can release the spring 1802. Releasing the spring 1802 can cause rotation of the crankshaft 1804. With the rotation of crankshaft 1804 in the direction of arrow 2000, the crankpin 1906 rotates around the rotating center 2002 and pushes the first end 1912 of the connecting rod 1808 to move downwardly parallel to the axis of the applicator 1800. The connecting rod 1808 pushes the piston 1810 to move downwardly through the movement of the second end 1914 of the connecting rod 1808. The downward movement of the piston 1810 drives the skin piercing device 6 through the skin. The downward movement of the piston 1810 may also the lancet shield 1816, and thus the wearable device 10, downward towards the skin of the user.

FIG. 21 illustrate the cross section view of the applicator 1800 during insertion at the lowest distal position. As illustrated in FIG. 21, the wearable monitoring device 10 is pushed against the user's skin and attach to the user's skin by an adhesive patch at the bottom of the wearable device 10. The skin-piercing device 6 is inserted into the user's skin. The connecting features 1923 on the lancet shield 1816 may be pushed downwardly along the movement of the lancet shield 1816. The downward movement of the lancet shield 1816 may stop when the edge of the holes 1909 on the applicator frame 1806 snap-fit into the neck 1927 of the snap-fit structure 1922 of the connecting features 1923 on the lancet shield. Thus, the lancet shield 1816 may be locked at the distal position.

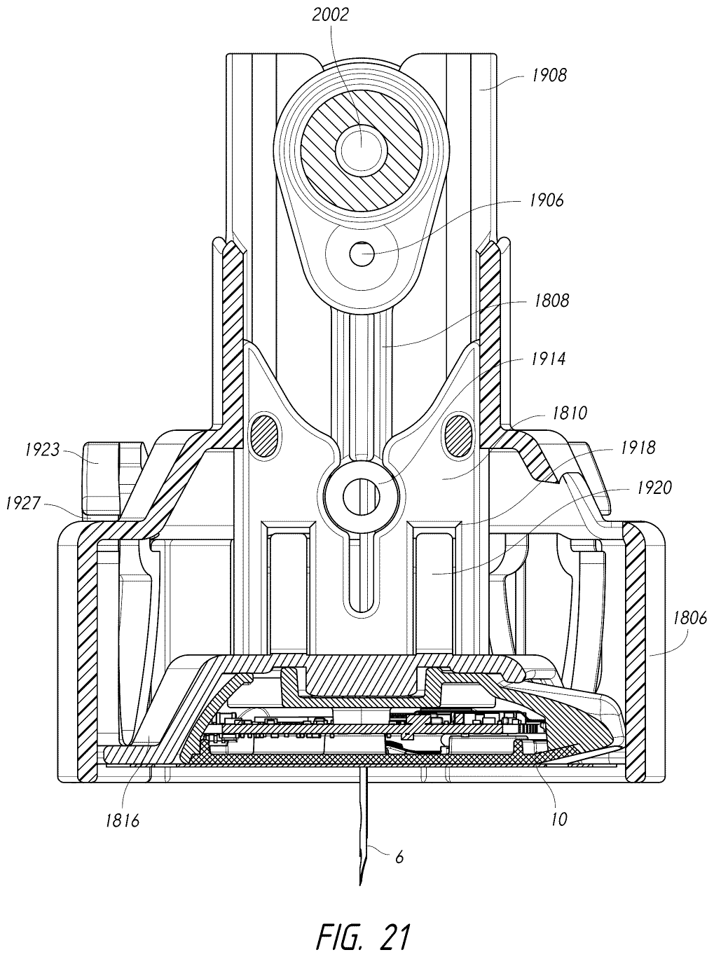

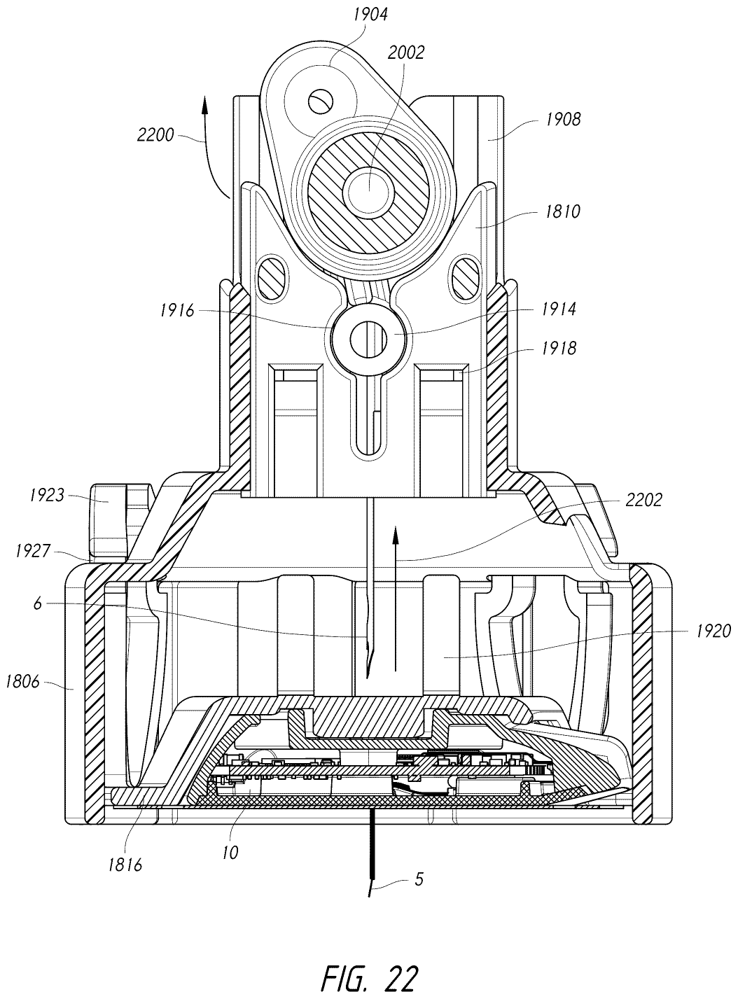

FIG. 22 illustrates the cross section view of the applicator 1800 during the retraction of the skin-piercing device 6 after the placement of the wearable analyte monitoring device 10 and insertion of the insertable portion 5 of the sensor. As illustrated in FIG. 22, as the rotation of the crankshaft 1804 continues in the direction of 2200, the crankpin 1906 pulls up the connecting rod 1808, which in turn pulls up the piston 1810. This retraction step is automatic as the crankshaft 1804 continues to rotate. The lancet shield 1816 is locked at the distal position such that it does not retract with the retraction of the piston 1810. The retraction of piston 1810 retracts the skin-piercing device 6 while the lancet shield 1816 stays at the distal position. Thus, the skin-piercing device 6 is hidden above the lancet shield 1816, such that the user is not vulnerable to the sharp tip of the skin-piercing device 6.

Prior to activation, the wearable analyte monitoring device 10 may be placed under the lancet shield 1816. Prior to activation of the device, the wearable analyte monitoring device 10 may be held under lancet shield 1816 by mechanical means, such as a low stick adhesive located between the wearable device and the piston. In some embodiments, a magnet may be placed within the piston 1810 and/or lancet shield 1816 that magnetically engages with a magnetic component within the wearable analyte monitoring device 10, such as a battery. The magnetic connection that secures the wearable device in place may be broken upon retraction of the piston 1810 releasing the wearable device 10 from the applicator 1800. In an alternative embodiment, the wearable device 10 may be retained under the lancet shield 1816 by a sterile cap key. The sterile cap key may align with one or more components on the bottom of the wearable device until removal of a housing bottom as discussed in the applicator designs above. Other features discussed above may be applicable in the design of applicator 1800.

In alternative embodiments, the shape of the different components of the applicator 1800 discussed may be different from the shapes illustrated in the figures, as long as the different components of the applicator can be assembled together to perform the function of the applicator 1800.

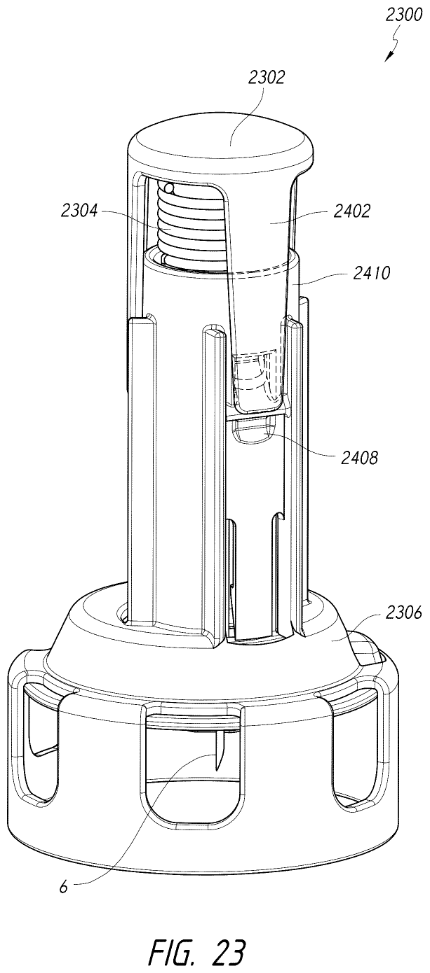

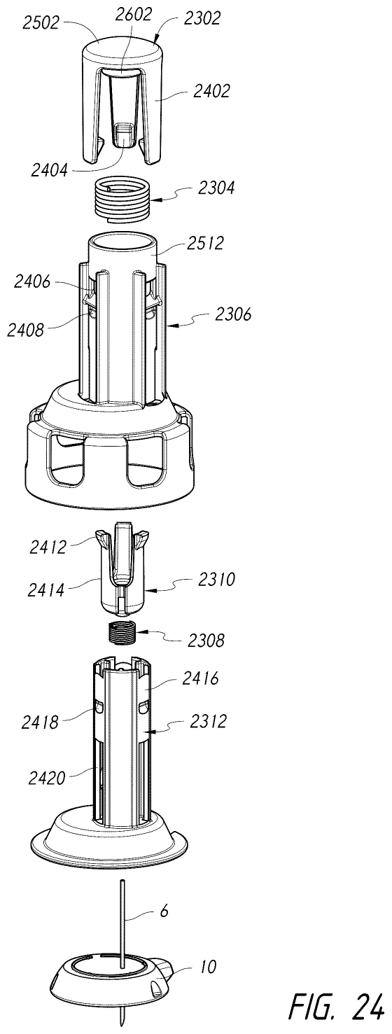

In an alternative applicator design, two springs are used. FIG. 23 illustrates another embodiment of the applicator 2300 absent the housing. FIG. 24 illustrates an exploded view of different components of the applicator according to the embodiment of FIG. 23. As illustrated in FIG. 24, the applicator 2300 may compromise a button 2302, an insertion spring 2304, an applicator frame 2306, a retraction spring 2308, a piston 2310, a skin-piercing device 6, and/or an applicator base 2312. In some embodiments, the wearable device 10 may be placed under the applicator base 2312 during assembly of the applicator 2300. In this design, the applicator 2300 includes one or more interlocking features to maintain the position of the relative components. Pushing an actuator like button 2302 releases one or more of the interlocking features, which allows an insertion spring 2304 to drive the applicator base 2312, and thus the wearable device 10. The applicator base 2312 also drives the piston 2310, which may be directly or indirectly coupled with the skin-piercing device 10. This design includes two separate springs for insertion and retraction. A separate retraction spring 2308 is responsible for retracting the skin-piercing device 6.

As illustrated in FIG. 24, the button 2302 may comprise a base portion 2502, and a cylindrical wall 2602 extending downwards from the base portion 2502 and one or more arms 2402 extending downwards from the base portion 2502. The arm 2402 may include a snap-fit hook 2404 at the end of each arm 2402. In some embodiments, the radius of the cylindrical wall 2602 is smaller than the radius of the base portion 2502. The piston 2310 may comprise piston arms 2414 extending from the bottom of the piston 2310. Each of the piston arms 2414 can include a piston snap-fit hook 2412 at the end of each arm 2414. The piston 2310 further comprises a receiving end to connect with the skin-piercing device 6 through a chuck 12 as discussed in the embodiments above.

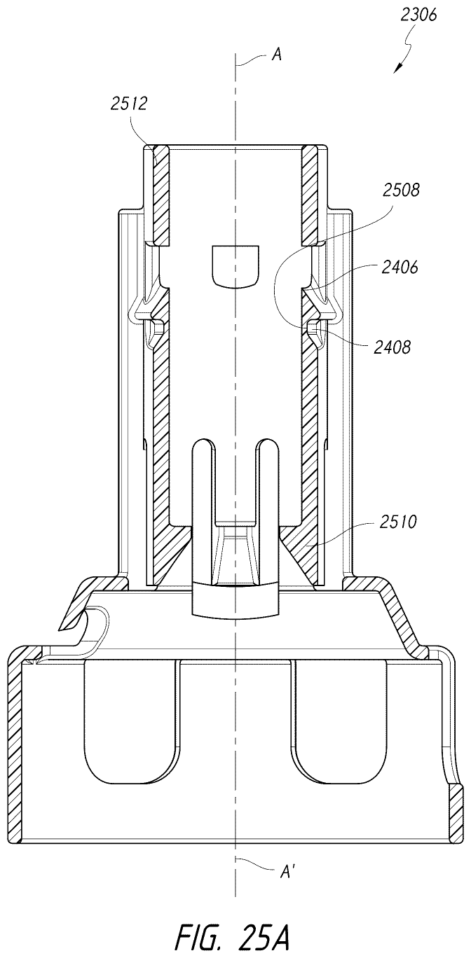

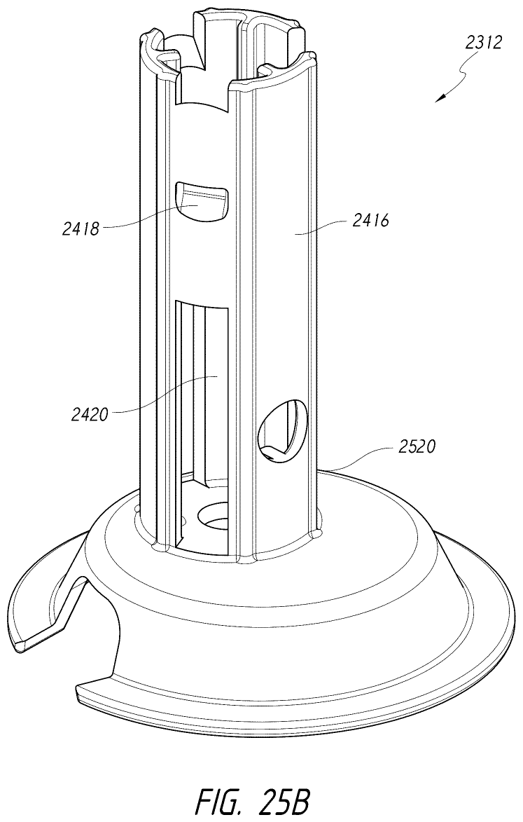

FIG. 25A illustrates the cross section view of an applicator frame 3608. As illustrated in FIG. 25A, the applicator frame 2306 may comprise a frame tube portion 2512 comprising one or more pairs of snap-fit recesses 2508. Each of the pair of snap-fit recesses 2508 comprises a first snap-fit recess 2504 and a second snap-fit recess 2506. The first snap-fit recess 2504 may be aligned with the second snap-fit recess 2506 in a direction parallel to the axis AA′ of the applicator 2300. The first snap-fit recess 2504 may be a through hole, while the second snap-fit recess 2506 may not a through-hole. In other embodiments, both the first 2504 and the second snap-fit recesses 2506 are through holes. In yet other embodiments, neither of the first 2504 and the second snap-fit recesses 2506 is a through hole. Both of first and the second snap-fit recesses can mate with the snap-fit hook 2404 of the button 2302. The applicator frame 2306 further comprises snap-fit hooks aligned with each pair of the recesses 2508 vertically. In some embodiments, the snap-fit hook 2510 is on the inside surface of the frame tube portion 2512 of the applicator frame 2306.

FIG. 25B illustrates a perspective view of the applicator base 2312. As illustrated in FIG. 25B, the applicator base 2312 comprises a housing base 2520 and an applicator base tube portion 2416 extending from the housing base 2520. The tube portion 2416 comprises snap-fit recesses 2418 and slots 2420 aligned with each of the snap-fit recess 2418 vertically. In some embodiments, the snap-fit recess 2418 matches with the snap-fit hook 2404 in the button 2302.

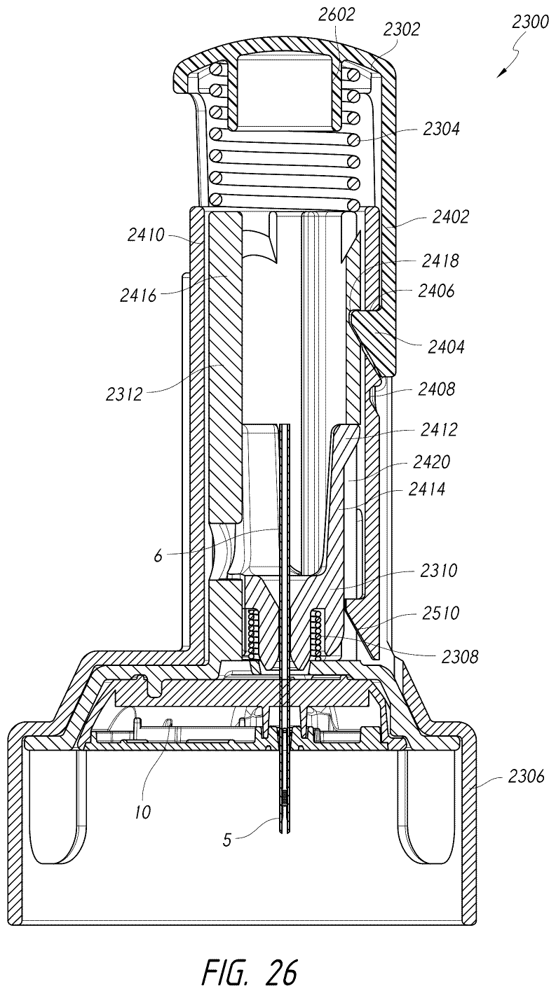

FIGS. 26-29 illustrate the cross section views of applicator 2300 during the insertion and retraction of the skin-piercing device 6, and the placement of the wearable analyte monitoring device 10 against a user's skin.

FIG. 26 illustrates the cross section view of the applicator 2300 at an initial starting position. The applicator 2300 includes one or more interlocking features, for example snap-fit features, to maintain the relative positions of the components. In this position, the insertion spring 2304 and the retraction spring 2308 are maintained in a loaded position. For example, as shown in FIG. 26, the snap-fit hooks 2404 in the button 2302 snap-fit into both the first snap-fit recess 2406 of the applicator frame 2306 and the snap-fit recess 2418 of the applicator base 2312. Thus, the insertion spring 2304, the applicator frame 2306, and the applicator base 2312 are fixed relative to each other at the initial position. The snap-fit hooks 2510 of the applicator frame 2306 is placed in the slots 2420 of the applicator base 2312.

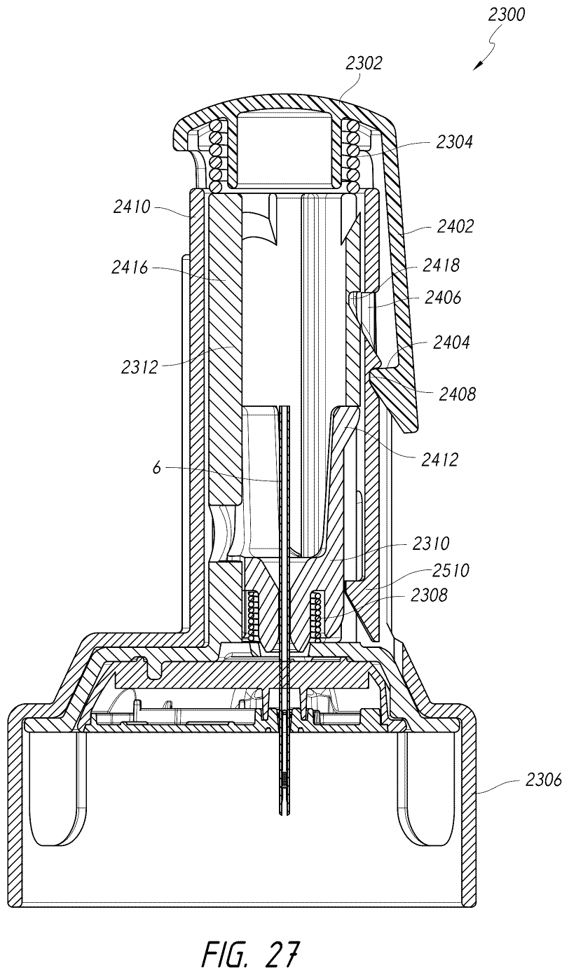

FIG. 27 illustrates a cross section view of the applicator 2300 after the button 2302 is pressed by a user. When a user presses down the button 2302, with the insertion spring 2304 is compressed, the snap-fit hook 2404 of the button 2302 moves down and is released from both the first snap-fit recess 2406 of the applicator frame 2306 and the snap-fit recess 2418 of the applicator base 2312. The button 2302 stops moving when the snap-fit hook 2404 of the button 2302 enters the second snap-fit recess 2408 of the applicator frame 2306. In this position, the button 2302 is interlocked with the applicator frame 2306, but not the applicator base 2312.

As the applicator base 2312 is not interlocked with the button through the snap-fit recess 2418 and snap-fit hook 2404 with the applicator frame 2306, the compressed insertion spring 2304 expands to push the applicator base 2312 downwardly, which pushes the wearable device 10 downwardly against the user's skin. The applicator base 2312 also pushes the piston 2310 to move downwardly to insert the skin-piercing device 6 into the skin.

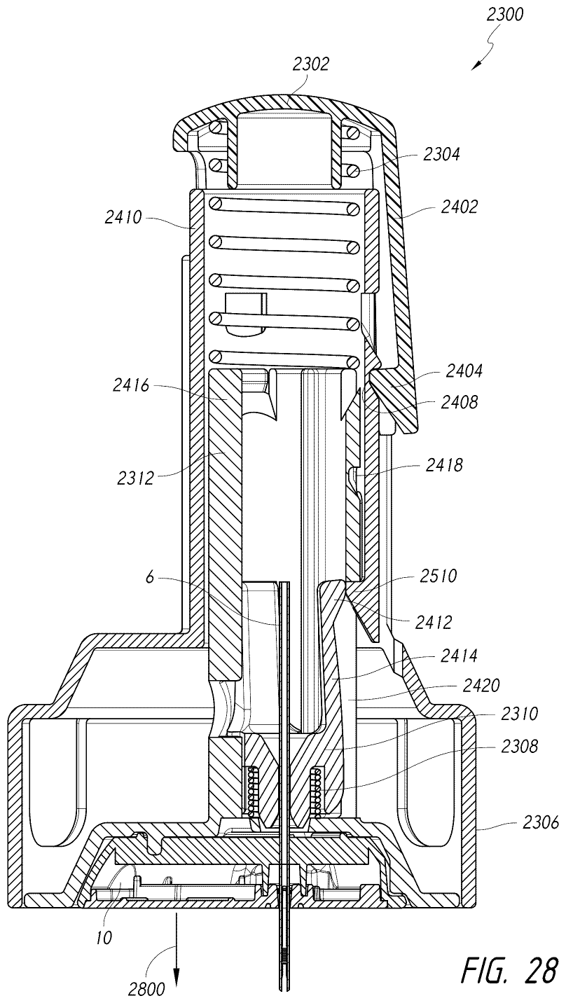

FIG. 28 illustrates the cross section view of a distal position of the applicator 2300. As illustrated in FIG. 28, the applicator base 2312 pushes the piston 2310 to the lowest position. The downward movement of the applicator base 2312 and the piston 2310 stops when the snap-fit hook 2510 of the applicator frame 2306 reaches the end of the slot 2420 of the applicator base 2312. The snap-fit hook 2510 of the applicator frame 2306 pushes the snap-fit hook 2412 of the piston 2310 inward and inside of the tube portion 2416 of the applicator base 2312. In this position, the piston 2310 is able to move relative to the applicator base 2312, which allows the compressed retraction spring 2308 may expand.

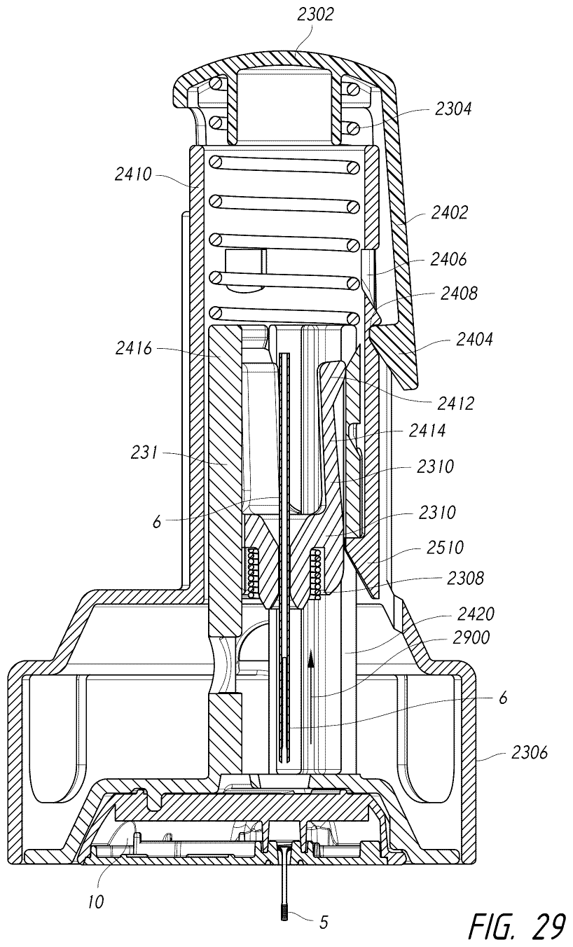

FIG. 29 illustrates the cross-section view of the applicator 2300 during the retraction of the skin-piercing device 6. As illustrated in FIG. 29, after insertion, the insertable portion 5 of the sensor stays in the user and the wearable device 10 stays on the user's skin. During retraction, the expansion of the retraction spring 2308 pushes the piston 2310 to move upward along the inside surface of the applicator base tube portion 2416 of the applicator base 2312. The skin-piercing device 6 retracts with the piston 2310. The applicator base 2312 stays at the distal position and does not move. Thus, the skin-piercing device 6 is hidden above the applicator base 2312, such that the user is not vulnerable to the sharp tip of the skin-piercing device 6.

Prior to activation, the wearable analyte monitoring device 10 may be placed under the applicator base 2312. Prior to activation of the device, the wearable analyte monitoring device 10 may be held under applicator base 2312 by mechanical means, such as a low stick adhesive located between the wearable device and the piston. In some embodiments, a magnet may be placed within the piston 2310 and/or applicator base 2312 that magnetically engages with a magnetic component within the wearable analyte monitoring device 10, such as a battery. The magnetic connection that secures the wearable device 10 in place may be broken upon retraction of the piston 2310 releasing the wearable device 10 from the applicator 2300. In an alternative embodiment, the wearable device 10 may be retained under the applicator base 2312 by a sterile cap key. The sterile cap key may align with one or more components on the bottom of the wearable device until removal of a housing bottom as discussed in the applicator designs above. Other features discussed above may be applicable in the design of applicator 2300.

In alternative embodiments, the shape of the different components of the applicator 2300 discussed may be different from the shapes illustrated in the figures, as long as the different components of the applicator can be assembled together to perform the function of the applicator 2300.

Advantageously, the two-spring applicator design may solve the retraction interruption issues caused by skin bulging. During the insertion of the skin-piercing device, the skin-piercing device may stop moving downward because a single spring may be not strong enough to overcome the force caused by the excessive skin bulging, although it has not reached the lowest distal position. With one spring and one cam design, the skin-piercing device cannot be retracted in this situation because the retractor has not reached the lowest point of the groove on the cam and the retractor cannot retract before it reaches the lowest point of the groove. In contrast, after the downward force applied to the applicator is released, the retraction spring expands and the retraction of the skin-piercing device may start.

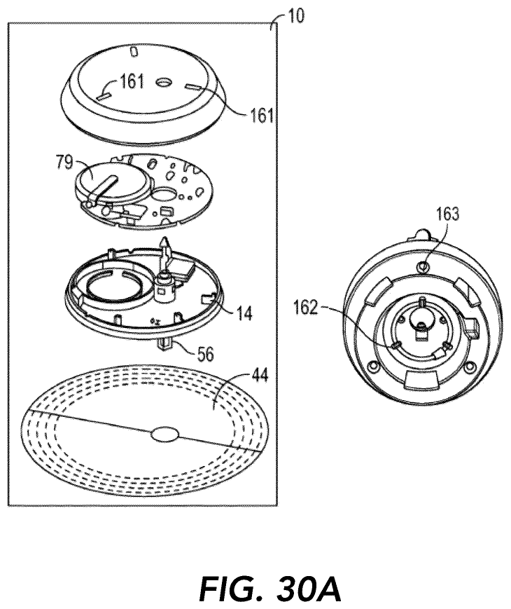

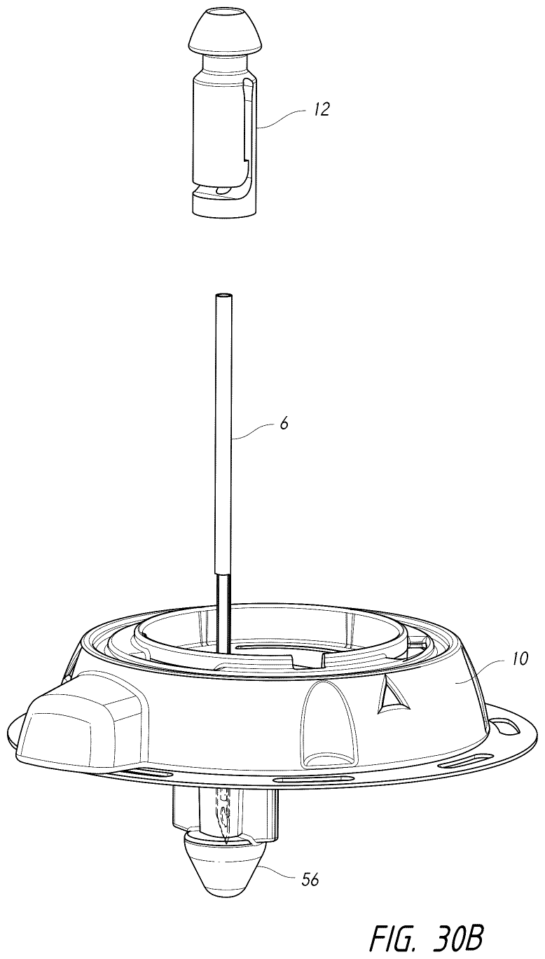

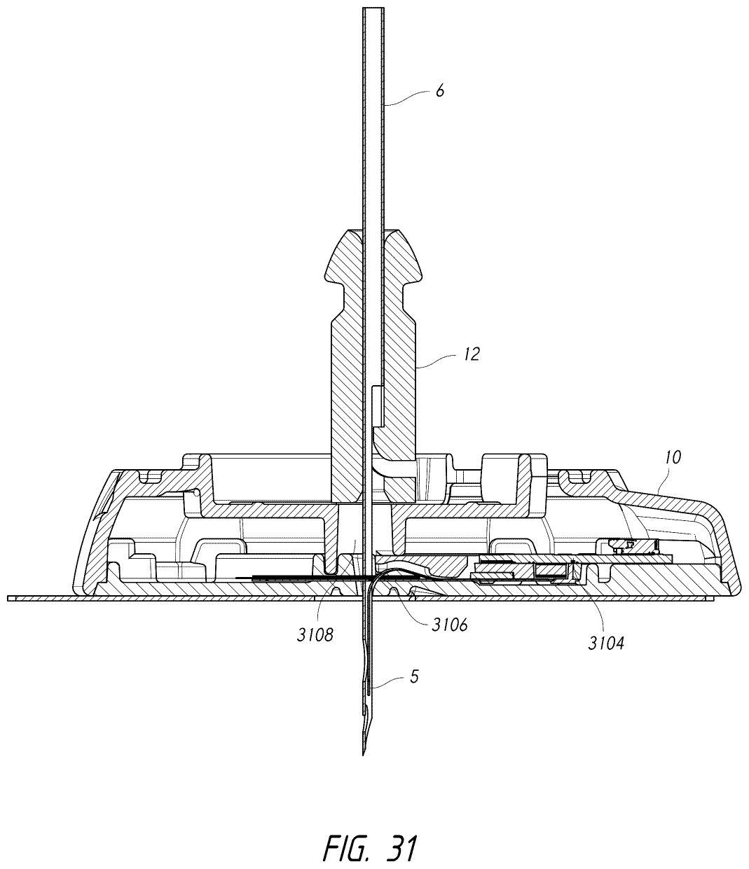

FIG. 30 illustrates the wearable analyte monitoring device 10 and the skin-piercing device 6 according to some embodiments. FIG. 31 illustrates the cross-section view of the wearable analyte monitoring device 10 and the skin-piercing device 6 according to some embodiments.

As illustrated in FIGS. 30 and 31, the wearable analyte monitoring device 10 may comprise a through hole 3108 for the skin-piercing device 6 to move up and down independent of the wearable analyte monitoring device 10. In some embodiments, the wearable analyte monitoring device 10 may comprise removable electronics 3104 housed inside of the wearable device 10. In some embodiments, at least a portion of the skin-piercing device 6 is hollow. In some embodiments, a lower portion of the skin-piercing device 6 is a half tube. In some embodiments, an insertable portion 5 of the sensor is placed into the half tube of the skin-piercing device 6. The wearable analyte monitoring device 10 and the skin-piercing device 6 may be configured to be used with any of the applicator embodiment discussed above. Further anti-rotation features that may be compatible with any of the above-described applicator embodiments are described in further detail below.

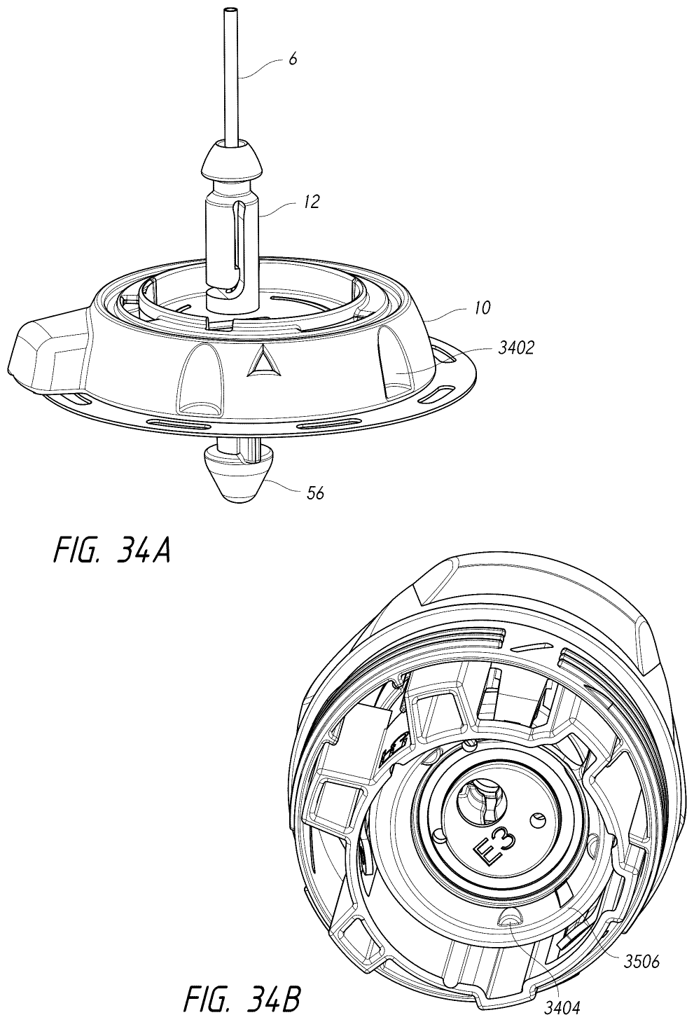

Air might be built up in the area between the user's skin and the wearable device in the applicator during the insertion. In any of the above-described embodiments, the applicator frame, piston, and/or the housing top may comprise vent holes, which provides an escape path to eliminate the volume of air pressure built up between the piston and the user's skin during insertion. The built up of air pressure may slow down the insertion speed. For example, as illustrated in FIG. 2, the housing top 2 may include one or more perforations 204. As illustrated in FIG. 12B, the applicator frame 30 may include one or more through holes 116 on the side wall. As illustrated in FIG. 34C, the piston base may comprise vent holes 163 providing an escape path to eliminate the volume of air distal to the piston base. In addition, the vent holes may be located on the bottom of the piston. These perforations 204 may act as vent holes to provide the escape path to eliminate the volume of air pressure.

In any of the above-described embodiments, the whole or substantially the whole applicator is made of plastic except the springs. In some embodiments, each component of the applicator, except the springs, is 3D-printed.

In any of the above-described embodiments, the analyte sensor applicator may be recycled and reused. In some embodiments, a replaceable inner cartridge may include the piston, sensor, and/or the skin-piercing device of any of the above described embodiments. The inner cartridge may be compatible with the sensor applicator. The inner cartridge may be disposed after usage and a new inner cartridge may fill the sensor applicator. In some embodiments, a kit may be provided with the reusable applicator and the replaceable inner cartridge. The kit may further include a tool to reload any spring in the applicator.

### Sterile Assembly

A wearable analyte monitoring device 10 may include a wearable housing, an analyte sensor assembly comprising an analyte sensor, an electronics components assembly, transducer, and/or battery. An adhesive pad may be optionally attached to the wearable device to affix the wearable analyte monitoring device 10 to the skin of the user. FIGS. 30A-30B illustrate different embodiments of a wearable analyte monitoring device 10. As illustrated in FIGS. 30A-30B, a wearable analyte monitoring device 10 may have a round disc shape or may further comprise a protrusion portion. The wearable analyte monitoring device 10 may comprise a battery 79 and other electronics.

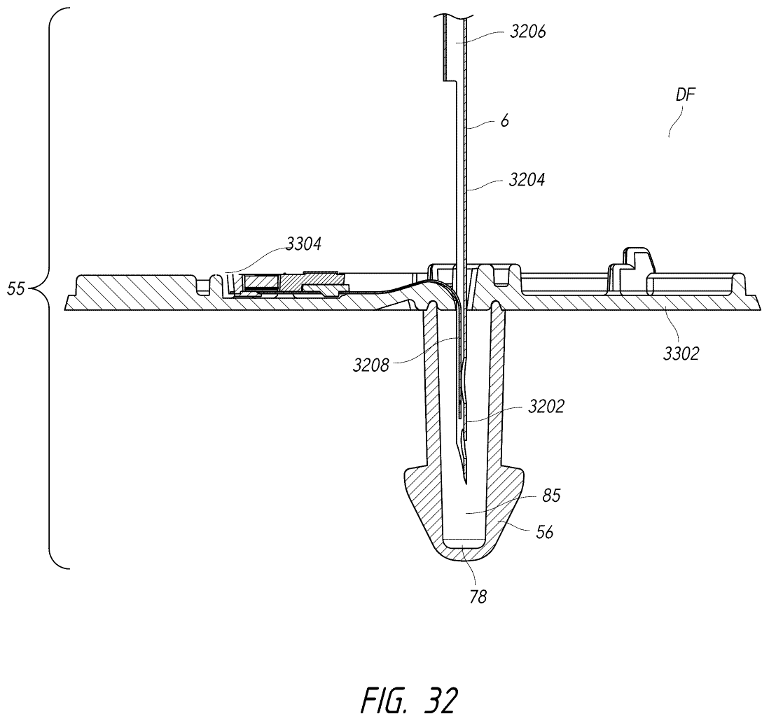

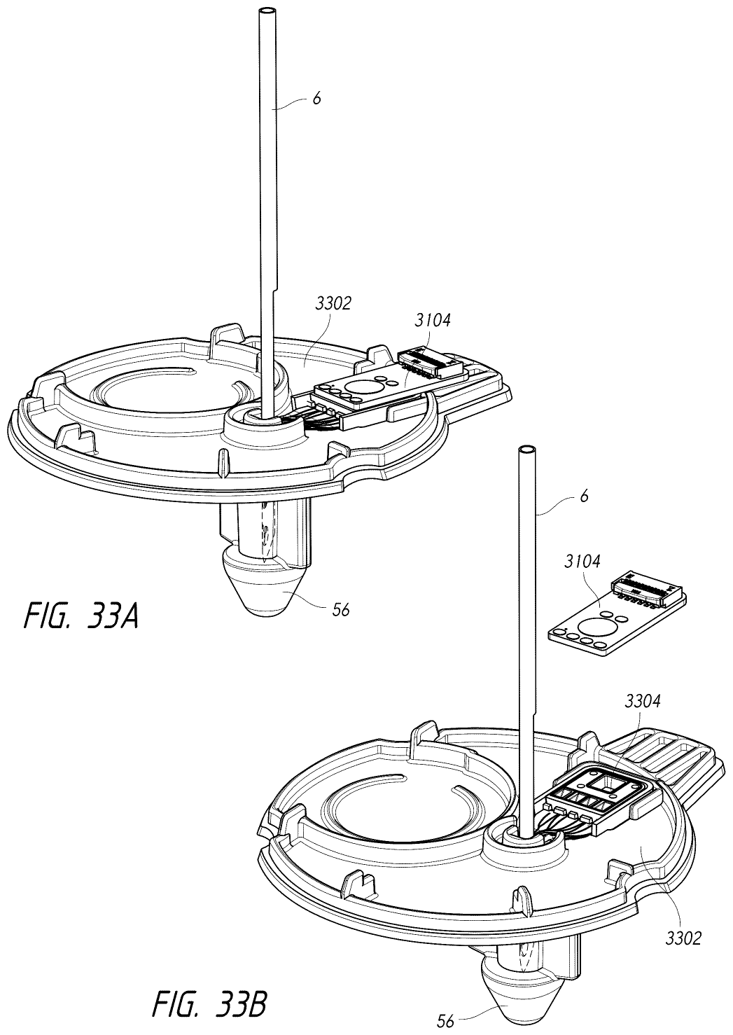

FIG. 32 illustrates a cross-section view of a sterile assembly 55 according to some embodiments. FIG. 33A illustrates a perspective view of the sterile assembly with the electronics 3104 attached to the sterile assembly 55. FIG. 33B illustrates a perspective view of the sterile assembly 55. In some embodiments, the sterile assembly 55 comprises a wearable device base 3302, a skin piercing device 6, and/or a sterile cap 56 sealing a lower portion of the skin piercing device 6 against the wearable device base 3302. The sterile assembly 55 may further comprise the optics portion 3304. As illustrated in FIGS. 33A and 33B, the electronics 3104 is removable and can be attached to the wearable device base 3302. In some embodiments, the wearable analyte monitoring device 10 may comprise a sterile subassembly 55 without the electronics 3104, the sterile assembly 55 may be sterilized prior to further manufacturing of the wearable analyte monitoring device 10. The sterile assembly 55 may remain sterilized until the user opens sterile cap 56 and insert the sensor into the user. After sterilization, the electronics 3104 may be added for final packaging. Effective methods for sterilizing the insertable components (such as, electron beam (E-BEAM) sterilization, or other radiation-based sterilization techniques) may be deleterious to electronic components of the analyte sensor assembly as illustrated in FIG. 32, insertable portions of the sensor 3102 and the skin-piercing device 6 are sealed in a small chamber 85, such as a sterile cap 56, which is sterilized prior to incorporation with other components of the wearable device during manufacturing. The insertable portion 3102 of the sensor may be wired to the wearable device base 3302 for the removable electronics. The insertable portion 3102 of the sensor may comprise a looped distal portion (discussed below with regard to FIG. 37) that may be mated to the tip of the skin-piercing device 6. The skin-piercing device 6 and the insertable portion of the sensor may be sterilize and placed in the sterile cap 56.

FIG. 34C illustrates another embodiment of wearable analyte monitoring device 10 which may be used with the applicators discussed above

The sterile cap 56 has an opening at a first end to receive the insertable portions into a chamber; the cap opening is sealed and coupled to the wearable device in a manner that a sterile barrier is achieved and maintained. An inert fluid 77 for example, argon gas, may fill the cap displacing environmental oxygen, such that the percentage of the oxygen present in the sterile cap 56 can be assumed to be zero during manufacture. A sealant is provided to seal the opening of the sterile cap 56 retaining the inert fluid within the chamber 85. The sterility of the insertable components is maintained within the chamber of the cap 56 until inserted into the user. By filling the sterile cover with an inert gas such as Argon during assembly the % oxygen present would be assured to be zero during manufacture.

A method is provided for confirming that a) sterile barrier has been established and b) the sterile barrier is maintained after further assembly. An oxygen sensing component 78 may be applied to, or added to, the chamber 85 of the cap prior to sealing. After assembly, the cap may be interrogated by a light source, such as a UV light source, for the presence of oxygen, an indication of leak in the sterile subassembly through which oxygen is entering the chamber. The oxygen sensing component 78 may comprise an oxygen sensing polymer, polymer laminate, or polymer matrix doped with a luminescent compound, such as an oxygen detecting dye. In some embodiments the dye is a luminescent dye. In some embodiments, the dye is a porphyrin dye, such as platinum tetrakis pentafluorophenyol porphyrin (pT-TFPP). Luminescent dyes (e.g., metallo derivatives) may emit a measurable signal dependent on the amount of oxygen present. In some embodiments a porphyrin dye is configured to reversibly bind to oxygen and to emit light when oxygen is bound A light source of a compatible or specified frequency, such as a UV light, may be used to interrogate the sterile cap for the presence or absence of fluorescence behavior that is indicative of the presence of oxygen. For example, if the oxygen sensing polymer emits bright red light after being interrogated by a UV light, there is no oxygen inside the sterile cap 56. If the red light is not bright, oxygen may be present inside of the sterile cap 56, such that the sensor and the skin piercing device need to be sterilized again. Where interrogation signals a breach of the sterile barrier, potential exposure of the insertable components within the chamber to a non-sterile environment may be assumed.

As illustrated in FIGS. 11A-11B, the inspection may be conducted through the port 54 at the bottom of the sterile cap key 35. To facilitate interrogation and visualization, the sterile chamber of the cap 56, or at least a portion of the sterile chamber may comprise a material that is optically transparent to sterilization (e.g., UV sterilization) and/or interrogation technique, such as a clear polycarbonate. In one embodiment, the sterile chamber comprises a one-piece cap that couples to the wearable base 10, for example, by welding or other sealing method, to form a sealed joint. To further inhibit gas permeability, at least a portion of the cap 56 may be coated with a non-gas permeable material. Alternatively, the cap may be covered by a second cap comprising a non-gas permeable material that may be coupled to the wearable base 10, or may be coupled directly to the first sterile cap. Where the second cap is molded into the wearable base, a break joint may be provided to facilitate separation of the cap from the wearable base prior to affixing to a user. In some embodiments, the inspection may be carried out from the side of the applicator before assembling. In some embodiments, the inspection may be carried out from an inspection port located at a side surface of the bottom housing.

In other embodiments the cap may comprise two pieces, wherein a first piece is molded with the wearable base, or integrated into, or coupled with, the wearable base. A second part of a two-part sterile chamber may be coupled to the first part by known means such as welding, adhering, press-fitting, screwing, and the like.

In some embodiment shape of the sterile cap 56 may comprise cylinder shape, ball shape, rectangular shape, or any other shape. In some embodiments, the sterile cap 56 may comprise other shapes configured to be inserted into the opening of the housing bottom. In some embodiments, the sterile cap 56 may be engaged in the sterile cap key 35 as discussed below. In some embodiments, the sterile cap 56 may comprise other features to aid in the engagement of the sterile cap with the sterile cap key 35.

Examples of analyte sensors and oxygen sensing components suitable for use include, but are not limited to materials disclosed in commonly owned U.S. patent application Ser. No. 16/490,118, filed Feb. 28, 2019, entitled ANALYTE SENSORS AND METHODS OF MANUFACTURING ANALYTE SENSORS, and U.S. patent application Ser. No. 16/193,305, entitled SYSTEMS AND METHODS FOR CONTINUOUS HEALTH MONITORING USING AN OPTO-ENZYMATIC ANALYTE SENSOR″, filed Nov. 16, 2018, the contents of which is incorporated by reference herein in its entirety for all purposes.

As illustrated in FIGS. 11A and 11B, the analyte sensor applicator housing bottom 3 further comprises an inspection port 54 to inspect the sterile cap 56 for the presence of a sterile environment. The inspection port 54 in the applicator housing aligns with an optically transparent portion of the sterile cap after assembly. The inspection port 54 may be incorporated as hole, or other optically transparent component at any location on the housing that aligns with an optically transparent portion of the sterile subassembly 55. The sterile barrier may also be interrogated through the final packaging materials. In the embodiment of FIGS. 11A and 11B, the inspection port is within the sterile cap key 35 that secures the sterile subassembly 55 to the housing bottom 3. In the embodiment of FIGS. 11A and 11B, the inspection port 54 is visible through a transparent portion or hole in a sterile cap key 35 located in the housing bottom 3.

In some embodiments, the sterile assembly may be placed in a low oxygen environment to reduce the oxygen permeated into the sterile assembly. In some embodiments, the sterile assembly may be placed in an evacuated space which may reduce some effects caused by temperature fluctuations.

In other embodiments, an applicator as discussed above may be sterilized after being assembled with the wearable analyte monitoring device and the skin piercing device. In some embodiments, the applicator as discussed above may be sterilized only once after being assembled with the wearable analyte monitoring device and the skin piercing device, such that no prior sterilization of some components may be needed. In those embodiments, sterilization with methods not damaging the electronics may be utilized. In some embodiments, sterilization steps may be taken during the manufacturing and assembling of the applicator.

### Rotation-Preventing Features

In any of the above-described embodiments, to prevent the relative rotation between the wearable analyte monitoring device and the component in direct contact with the wearable analyte monitoring device, such as a piston, the wearable analyte monitoring device may include rotation-preventing features that match with the rotation-prevention features on the surface of a component in direct contact with the wearable analyte monitoring device. Without such rotation-preventing features, when a user rotates the housing bottom to remove the housing bottom before activating the applicator, the wearable analyte monitoring device may rotate relative to the piston or other contacting component because the sterile cap key connects the housing bottom to the sterile cap, which is connected to the base of the wearable analyte monitoring device, such that the wearable analyte monitoring device may rotate with the rotation of the housing bottom. The rotation of the wearable monitoring device may cause damage to the skin-piercing device and/or an adhesive patch at the bottom of the wearable monitoring device for attaching the device to the skin. Advantageously, with the matching rotation-preventing features on the wearable analyte monitoring device and the piston, the rotation of the housing bottom only causes the rotation and removal of the sterile cap and does not cause rotation of the wearable analyte monitoring device.

FIGS. 34A and 34B illustrate examples of the matching rotation-preventing features on the wearable analyte monitoring device and the piston or other contacting component. As illustrated in FIGS. 34A and 34B, in some embodiments, the outer surface of housing of the wearable analyte monitoring device 10 may comprise dimples or concave features 3402 and the bottom surface of the piston may comprise matching protrusion or convex features 3404. In some embodiments, the features 3402 may be protrusion or convex features and the matching features 3404 may be dimple or concave features. In some embodiments, the piston or the wearable analyte monitoring device comprises 1, 2, 3, 4, 5, 6, 7, 8, 9, 10, 15, 20, or any number therebetween, rotation-preventing features. In some embodiments, the shape and size of the matching features on the piston and the outer surface of the housing of the wearable analyte monitoring device are not limited.

FIG. 34C illustrate another example of the matching rotation-preventing features on the wearable analyte monitoring device 10 and the piston or other contacting component. As illustrated in FIG. 34C, the outer surface of housing of the wearable analyte monitoring device 10 comprises dimples or protrusions 161 may be provided to a surface of the wearable device, and mating structures 162, for example, in the form of protrusions or dimples, may be provided to the piston base 50 to hold the wearable device in place. By holding the wearable device in position, the adhesive patch 44 will be held in place as well. In a further embodiment, the piston base comprises vent holes 163 providing an escape path to eliminate the volume of air distal to the piston base that is displaced during insertion, preventing sliding of the adhesive 44 or dimpling of the user's skin for better placement.

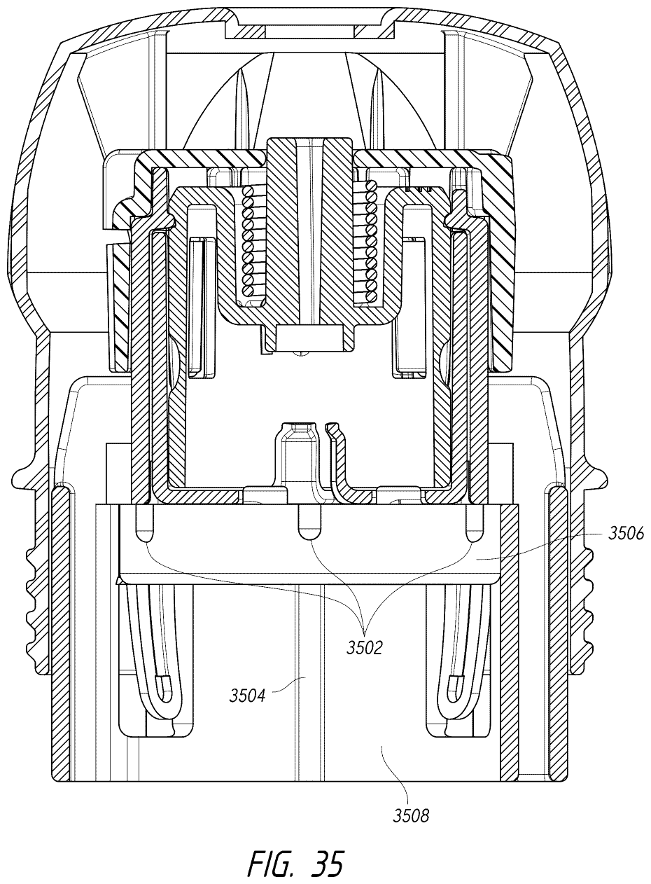

FIG. 35 illustrates features to prevent rotation of piston according to some embodiments. As illustrated in FIG. 35, the piston 3506 may comprise protrusion features 3502. The applicator frame 3508 may comprise vertical slides 3504 on the internal surface of the application frame. The protrusion features 3502 may fit in the slide 3504, such that the piston may only move in a vertical direction parallel to the direction of the slide 3504. These anti-rotation features between the piston and the applicator frame may be included in any of the above-described applicators.

### Sterile Cap Key

The applicator may comprise a sterile cap key. The sterile cap key may connect the sterile cap with the housing bottom. For example, the sterile cap key may include a sterile cap engaging portion that engages the sterile cap. The geometry of the engaging portion is configured to engage and hold the sterile cap against the base of the wearable device to keep the wearable device in place, such that an adhesive patch for attaching the wearable device to the user's skin is kept in position. The design of the sterile cap key and sterile cap can be configured to be applicable with any of the applicator design disclosed herewith.

The sterile cap key may be inserted into the opening of a bottom housing of the applicator. For example, the sterile cap key may sit in the opening of the housing bottom loosely. The sterile cap key may be connected to the bottom housing through a loose-fitting connection. Such loose fit may provide flexibility for re-aligning the housing bottom and the wearable device and eliminating stress that may causes the breach of the sterile barrier as the misalignment between the housing bottom and the wearable device may place stress on the joint between the sterile cap and the wearable device. In some embodiment, the connection between the housing bottom 3 and the sterile cap key is a pivot. In some embodiments, the sterile cap key may be connected to the bottom housing through snap fit.

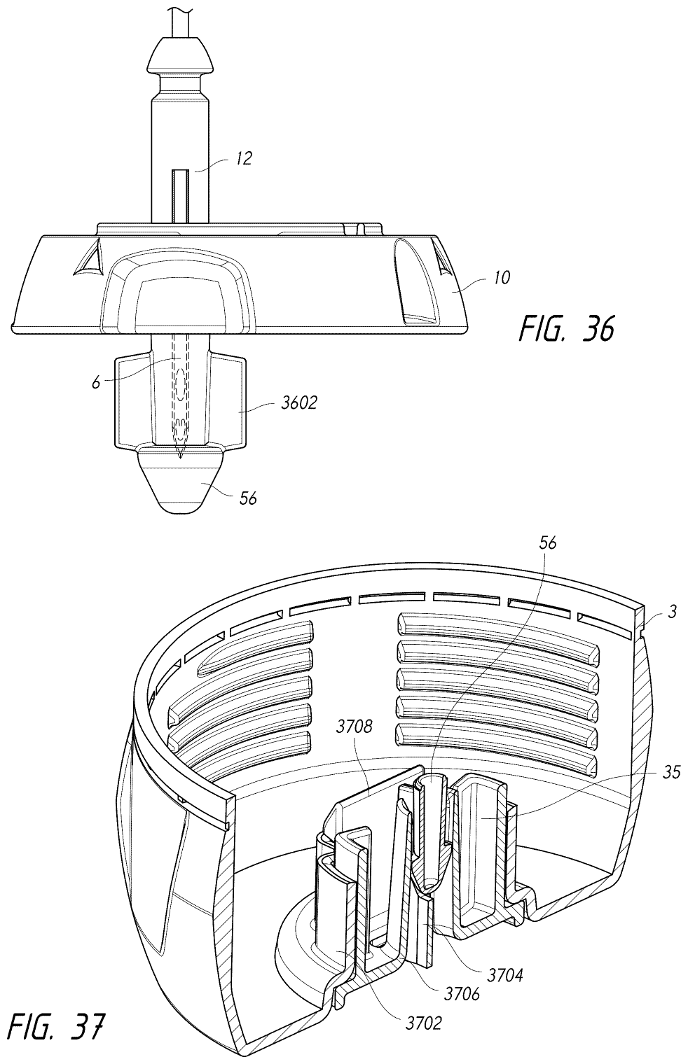

In some embodiments, the sterile cap key may facilitate the removal of the sterile cap with the removal of the bottom housing. FIGS. 36 and 37 illustrates an example of a sterile cap 56 and the sterile cap key 35. As illustrated in FIG. 36, the sterile cap 56 may comprise one or more pillars 3602 and/or a cone-shaped bottom 3604.

As illustrated in FIG. 37, the sterile cap key 35 may be inserted into the opening of the housing bottom 3 via snap-fit. For example, the sterile cap key 35 may include one or more tabs or cantilevered structures 3708 configured to form a snap-fit with the opening of the housing bottom 3. The sterile cap key 35 comprises a sterile cap engaging portion 3706 that engages with the sterile cap 56 when the housing bottom 3 is secured together with the housing top 2. The engagement between the engaging portion 3706 and the sterile cap 56 may be loose. The engagement between the engaging portion 3706 and the sterile cap 56 is not tight because a tight engagement may break the seal of the sterilized chamber. The sterile cap engaging portion 3706 may include a portion 3704 that pushes the sterile cap 56 against the bottom of the wearable device 10, such that the wearable device 10 stays in position.

When a user rotates or twists the housing bottom 3 to remove the housing bottom 3 from the applicator, the sterile cap key 35 rotates with the bottom housing 3, such that the engaging portion 3706 applies rotation force to the sterile cap 56. As the sterile cap 56 rotates with the sterile cap key 35, the joints between the sterile cap 56 the base of the wearable device 10 break and the sterile cap 56 may be removed with the rotation and removal of the housing bottom 3. The pillars 3602 of the sterile cap 56 allow for rotational torsion on the sterile cap without a tight engagement between the engaging portion 3706 and the sterile cap 56. The cone-shaped bottom 3604 allows for a guided entry of the sterile cap key and keeps the sterile cap connected to the housing bottom during removal. In some embodiments, the sterile cap key may be a square or rectangular shape. In some embodiment, the sterile cap key may be any other shape that can rotate with the rotation of the housing bottom. In some embodiments, the geometry of the sterile cap may be any other shape and the geometry of the engaging portion 3706 is adjusted accordingly. Advantageously, such design may simplify the steps for the user to use the applicator.

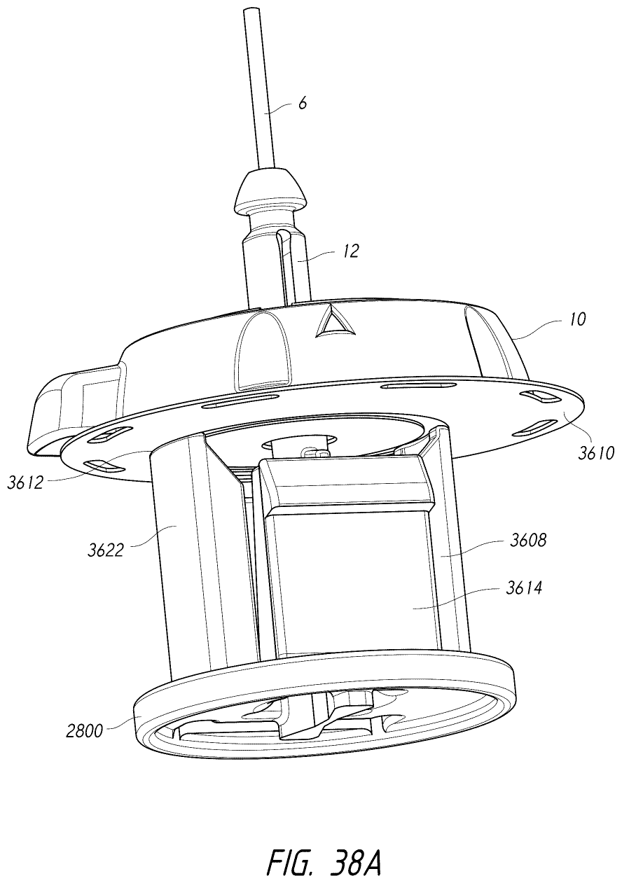

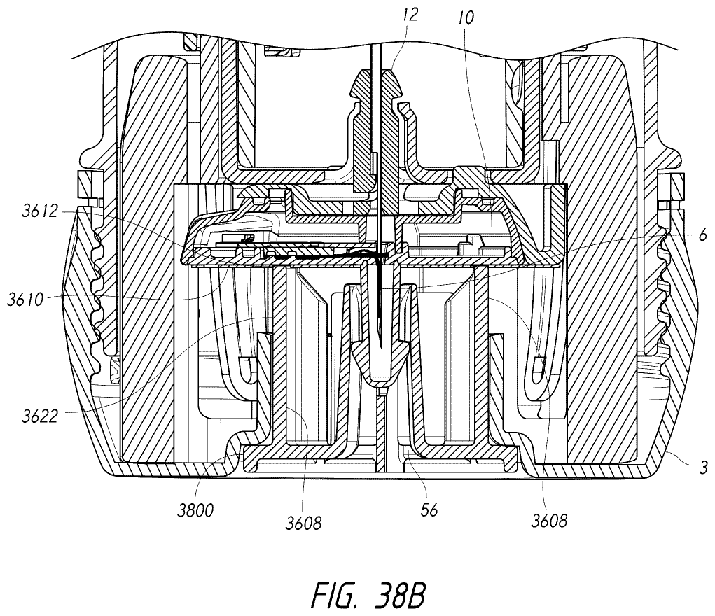

The sterile cap key may comprise one or more pillars which are in contact with the bottom of the wearable device. FIGS. 38A and 38B illustrates a sterile cap key 3800 according to some embodiments. The sterile cap key 3800 shown in FIGS. 38A-38B may include any of the features shown in FIG. 37, for example snap-fit features for interlocking with the housing. As illustrated in FIG. 38A, sterile cap key may include one or more pillars 3602 and interlocking tabs 3614. The pillars 3602 may extend from the bottom of the sterile cap key 35 and are in direct contact with the bottom of the wearable device 10 through the holes 3612 of the adhesive patch 3610. The pillars 3602 may extend beyond the interlocking tabs 3614. As illustrated in FIG. 38B, the pillars 3602 may provide support for the wearable device 10 to keep the wearable device 10 in place. Such support may reduce the engagement force between the sterile cap and the engagement portion of the sterile cap key. In addition, such support will prevent the wearable device from damage during transportation and storage.

### Adhesive Patch

In some embodiments, an adhesive patch may be used to attach the wearable device base to the user's skin. As illustrated in FIG. 38A, the adhesive patch 3610 is attached to the bottom of the wearable device 10. In some embodiment, the adhesive patch may be attached to the bottom of the wearable device by methods such as adhesive, laser welding, heat staking, etc. In some embodiment, the welded area or bonded area for attaching the adhesive patch to the wearable device may cause skin irritation. In some embodiments, a second adhesive patch may be attached to the first adhesive patch to avoid the contact between the user's skin and the bounded or welded area. In some embodiments, a hypoallergenic material is applied directly to the bonded or welded area to avoid the contact between the skin and the welded or bonded area. Additional information can be found in U.S. application Ser. No. 16/470,839, entitled “ADHESIVE SYSTEMS HAVING AN AGGRESSIVE ADHESIVE OUTER RING ALONG ITS BOTTOM PERIMETER AND HAVING A LOW EFFECTIVE MODULUS OF ELASTICITY,” which is incorporated here in its entirety.

### Skin-Piercing Device

As described above, each of the applicators include a skin-piercing device. The skin-piercing device may take on any of the configurations described below. The skin-piercing device can be used to insert a sensor or an insertable portion of a sensor to a right position under a patient's skin, for example into the interstitial fluid of the user. In some embodiments, the sensor is an analyte sensor for a continuous glucose monitoring system comprising the wearable analyte monitoring device, but may be used to sense any analyte. The analyte sensor may include an insertable portion of the sensor configured to be placed inside of the user, an optical assembly, and a wire connecting the insertable portion of the sensor and the optical assembly. In some embodiments, the optical assembly comprises electronics.

The skin-piercing device may pierce the user's skin to implant the insertable portion of the sensor into the user. The skin-piercing device can be configured to retract from the user and leave the insertable portion of the sensor inside of the user after retraction.

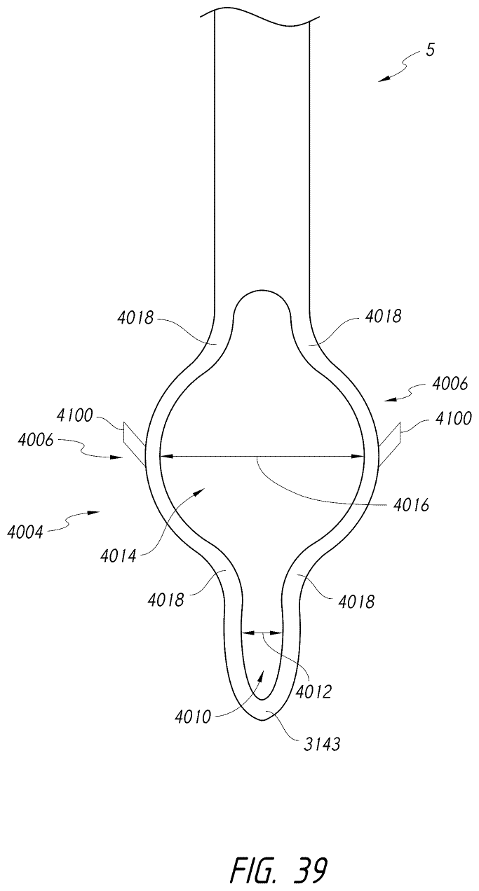

FIG. 39 illustrates a top-down view of a distal portion of a sensor. As shown in FIG. 39, the distal portion of the insertable portion 5 of the sensor can include a looped distal portion 4004 at the distal end of the insertable portion of the sensor 5 to attach the insertable portion of the sensor 5 to the skin-piercing device 6. The sensor looped distal portion 4004 can include a first opening 4010 that is adjacent to the loop tip portion 3143 with a maximum first width 4012 and a second opening 4014 disposed between the proximal end 4008 and the first opening 4010. The second opening 4014 has a maximum second width 4016 that is greater than the maximum first width 4012. The first opening 4010 and the second opening 4014 are contiguous.

With continued reference to FIG. 39, the looped distal portion 4004 may further comprise one or more protrusions 4100. For example, the protrusions 4100 may be positioned on the second opening 4014. The protrusions 4100 may extend from the outer rim of the second opening towards the proximal end of the sensor. The protrusion 4100 may be angled relative to the axis of insertion. These protrusions 4100 may aid in the unloading of the sensor looped distal portion 4004 from the skin-piercing device and also may help in anchoring of the sensor in subcutaneous tissue.

The looped distal portion 4004 includes sensor looped transition portions 4018 (a) between the first opening 4010 and the second opening 4014 and (b) between proximal end 4008 and the second opening 4014 of the sensor looped distal portion 4004, that are thicker than the other portions of the sensor transmission element 4006. As discussed in more detail below, the thicker portions of the sensor looped transition portions 4018 aid in the unloading of the sensor looped distal portion 4004 from the skin piercing device 6 and also helps in anchoring of the sensor in subcutaneous tissue. After implantation, looped distal portion 4004 along with the insertable portion of the sensor 5, provides the required interstitial fluid information to the optical assembly comprising the electronics 3104 and hence, the analyte sensors of the embodiments of the present invention.

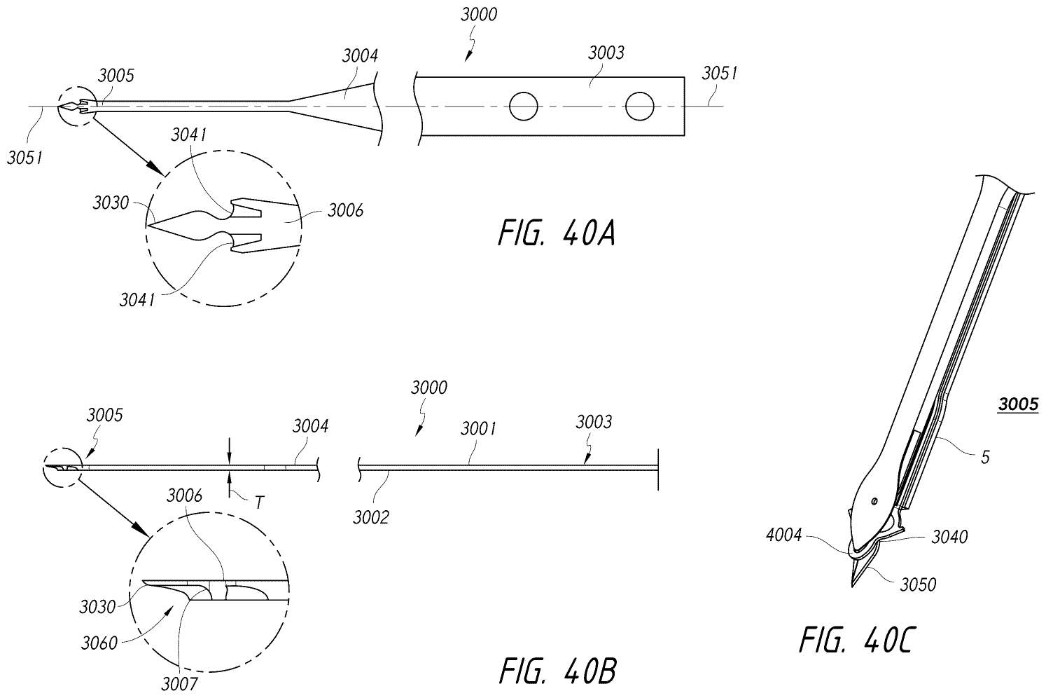

The skin-piercing device may include a lancet. FIGS. 40A-40C illustrate the design of the lancet/insertion structure 3000 according to some embodiments. As depicted in FIG. 40A and FIG. 40B, lancet/insertion structure 3000 according to some embodiments may comprise a substantially planar, non-rigid, non-frangible, elongate member having a proximal portion 3003, an intermediate portion 3004, a distal portion 3005 for piercing the skin and a longitudinal axis 3051.

FIG. 40C illustrates a design of the proximal portion 3003 according to some embodiments. The distal portion 3005 may include a first surface 3006, a second surface 3007 that is substantially opposite the first surface 3006, and a tip 3030. In order to cut through the skin and subcutaneous tissue during insertion, the distal portion 3005 includes at least one cutting surface/edge 3050. This cutting surface 3050 can be, for example, a positive convex surface that forms a cutting surface/edge. The distal tip portion may include a plurality of cutting surfaces 3050 that can be adjacent to the distal portion first surface 3006 and/or the distal portion second surface 3007 or that can be disposed between the distal portion first surface 3006 and the distal portion second surface 3007. The distal portion 3005 can include one or more insets or recessed portions 3040 that extend between the first surface 3006 of the distal portion 3005 and the second surface 3007 of the distal portion 3005. The one or more insets or recessed portions 3040 are designed to receive at least a portion of the looped distal portion 4004 located at the distal end of the insertable portion of the sensor 5 to be inserted/implanted into the skin and can be, for example, circular or curvilinear. More information regarding this embodiment can be found in application Ser. No. 15/754,271, entitled “SYSTEMS AND METHODS FOR CONTINUOUS HEALTH MONITORING USING AN OPTO-ENZYMATIC ANALYTE SENSOR,” which is incorporated here in its entirety.

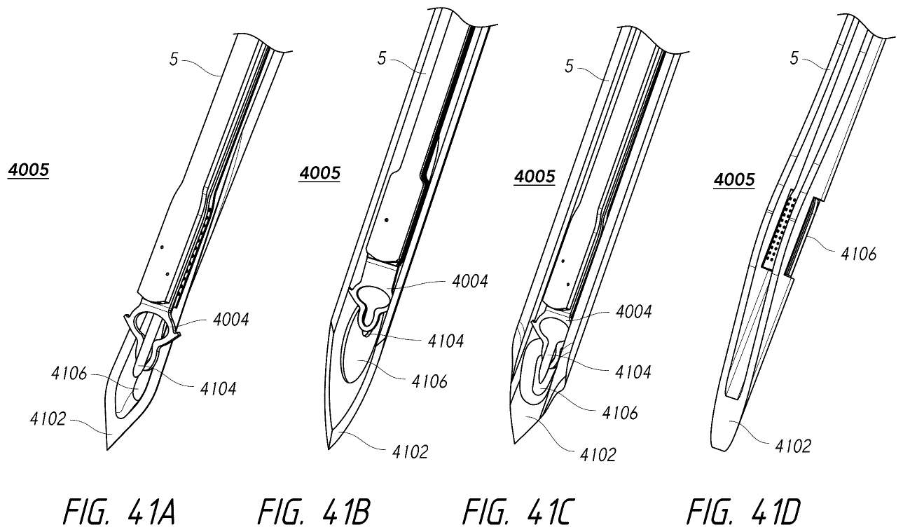

In some embodiments, the skin-piercing device may include a catheter. The skin-piercing device may be rigid. With reference to FIG. 32, the skin-piercing device 6 may include a distal portion 3202 for piercing the skin that may include any of the designs shown in FIGS. 40A-41D. The skin-piercing device may include a partially enclosed body 3204 for housing a wire 3208. The skin-piercing device may include a proximal portion 3206 for connecting the skin piercing device 6 to a driving mechanism such as the piston of any of the above-described applicators. As illustrated, the proximal portion 3206 may be tube-shaped or a rod without a through lumen. The partially enclosed body 3204 enables the insertable portion of the sensor 5 to be fixed to the distal portion 3202 of the skin-piercing device 6 and house the sensor wire 3208 before insertion. As the partially enclosed body 3204 is not fully enclosed, the retraction of the skin piercing device 6 will not interfere with the sensor wire 3208 after insertion.

FIGS. 41A-41D illustrate various designs of the distal portion 4105 of the skin-piercing device 6. As illustrated in FIGS. 41A-41C, the skin-piercing device 6 may comprise a cutting tip 4102 for cutting open the skin, a loop hook 4104, and a through-hole 4106. Loop hook 4104 may extend into the through-hole 4106 from a proximal end of through-hole 4106. In some embodiments, the first opening 4010 of the looped distal portion 4004 of the sensor may be placed on the loop hook 4104 for insertion. The through-hole 4106 may be sufficiently large to not interfere with the placement of the looped distal portion 4004 of the sensor during the retraction of the skin-piercing device 6. After insertion, as the skin-piercing device retracts, the looped distal portion 4004 keeps the insertable portion of the sensor 5 in the inserted position. Advantageously, the skin-piercing device 6 may protect the distal end of the sensor during the insertion such that the insertable portion of the sensor would not be stuck on a top layer of the dermis during insertion, such that the sensor would not be in contact with user's skin during insertion. Additionally, no tension needs to be applied to the looped distal portion 4004 during assembling the device, such relaxation aid in the release and placement of the sensor during insertion. Moreover, the design of the skin-piercing device can be modified to accommodate changes to the sensor's length and width. In some embodiments, the skin piercing device can be manufactured via a stamping process. As illustrated in FIGS. 41A-41C, the through-hole 4106 may comprise a shape of an oval shape, square shape, rectangular share, or any other shape.

FIG. 41D illustrates a distal portion 4105 of a skin piercing device 6 and a loopless insertable portion of the sensor 5. As illustrated in FIG. 41D, the distal portion of the skin piercing device 6 may include one or more through-holes 4106. The through-hole 4006 may be a square or rectangular shape. The square shape shown in FIG. 41D may further reduce the sensors profile and aid in automated inspections and assembly of the device.

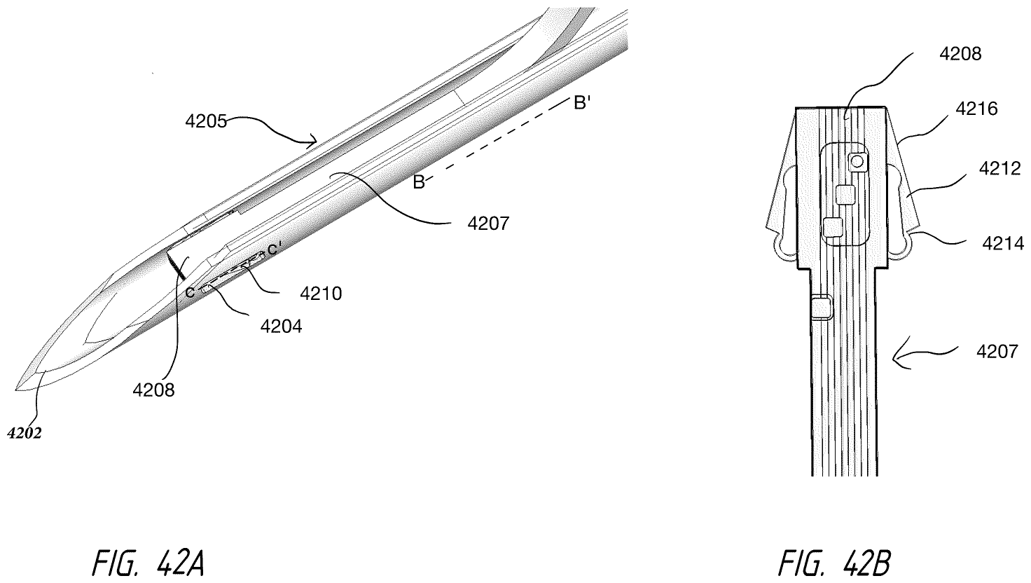

FIGS. 42A-42B illustrate another embodiment of the distal portion 4205 of the skin-piercing device 6 and the insertable portion of the sensor 4207. FIG. 42A illustrates a perspective view of a distal portion of the skin piercing device containing the insertable portion of the sensor according to some embodiments. FIG. 42B illustrates a top-down view of the insertable portion of the sensor according to some embodiments. As illustrated in FIG. 42A, the distal portion 4205 of the skin piercing device 6 may include a cutting tip 4202. The distal portion 4205 may include one or more sliding through-holes 4204, such as sliding slots. Each of the sliding through-holes 4204 may be disposed the sidewall of the distal portion of the skin piercing device 4205. A long axis CC′ of the through-holes may be parallel to the long axis of the skin-piercing device BB′. As illustrated in FIG. 42B, the insertable portion of the sensor 4207 may include a sensing tip 4208. The insertable portion of the sensor 4207 may include one or more sensor fasteners 4210. The sensor fasteners 4210 may be configured to be received in the sliding through-holes 4204 of on sidewalls of the distal portion 4205 of the skin-piercing device. The fasteners 4210 may be configured to attach the insertable portion of the sensor to the distal portion of the skin-piercing device 4207 during insertion. The fasteners 4210 may be configured to detach the insertable portion 4207 of the sensor from the distal portion of the skin-piercing device 4205 during retraction of the skin piercing device. In some embodiments, the fasteners 4210 may include a snap-fit connection. As illustrated in FIG. 42B, the fastener 4210 may comprise a sliding component 4212 and a fastener neck 4214. The sliding component 4212 may comprise an angled surface 4216 relative to the long axis BB′ of the skin piercing device. During the insertion, the fastener neck 4214 may be stuck at the edge of the sliding through-holes 4204 such that the insertable portion of the sensor is attached to the distal portion of the skin piercing device. The insertable portion of the sensor 4207 may be delivered to the expected position with the insertion of the skin-piercing device 6. During retraction, the skin piercing device 6 is retracted in a direction of 400. The sliding through-holes 4204 may slide against the angled surface 4216 of the sliding component. The sliding component 4212 may be compressed by the edge of the sliding though holes 4204 during retraction such that the fasteners 4210 may not be fixed to the sliding through holes, leaving the insertable portion of the sensor in the expected position.

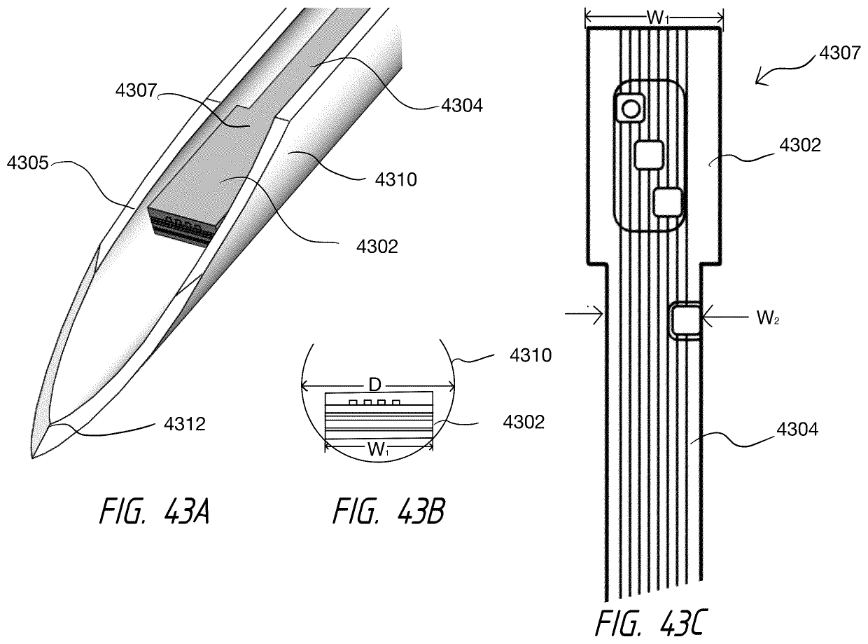

FIGS. 43A-43C illustrate another embodiment of the distal portion 4305 of the skin-piercing device 6 and the insertable portion of the sensor 4307. FIG. 43A illustrates a perspective view of the distal portion 4305 of the skin piercing device with the insertable portion of the sensor 4307 according to some embodiments. FIG. 43B illustrates the cross section view of the distal portion 4305 of the skin piercing device with the insertable portion of the sensor 4307. FIG. 43C illustrates the top-down view of the skin insertable portion 4307 of the sensor in FIG. 43A. The distal portion 4305 of the skin piercing device may comprise a hollow body 4310 with a diameter D. A portion of the body 4310 at the distal portion 4305 of the skin piercing device may be cut away such that the hollow body 4310 is arcuate but does not fully enclose the sensor. The hollow body 4310 may extend at least 180 degrees around the sensor but less than 360 degrees. The distal portion 4305 of the skin piercing device may comprise a cutting tip 4312. The insertable portion of the sensor 4307 may include a sensing tip 4302 and a connecting portion 4304. The sensing tip 4302 may comprise a first width W₁ and the connection portion 4304 may comprise a second width W₂. In some embodiments, the second width W₂ may be smaller than the first width W₁. In some embodiments, the second width W₂ may be the same as the first width W₁. The first width W₁ may be smaller than the diameter D of the distal portion of the skin piercing device. In this configuration, the position of the sensor is maintained within the distal portion 4305 of the skin piercing device until the hollow body 4310 is retracted. The first width W₁ may be configured to be placed in the distal portion of the skin piercing device 4305. A proximal portion of the skin piercing device (not shown) may include an arcuate body similar to the distal portion or a fully enclosed lumen.

### Terminology

While certain embodiments have been described, these embodiments have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result.

The scope of the present disclosure is not intended to be limited by the specific disclosures of embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

While certain embodiments have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

1. An analyte sensor applicator for inserting a sensor of a wearable analyte monitoring device, the analyte sensor applicator comprising:
an applicator housing;
a skin-piercing device and an insertable portion of the sensor;
a cam assembly within the applicator housing, the cam assembly comprising:
a cylindrical cam comprising a rotational axis and an external cam surface; and
a thrust barrel interfacing with the external cam surface, the thrust barrel comprising a bottom portion having a port therethrough for being coupled to the skin-piercing device; and
a piston in contact with the bottom portion of the thrust barrel,
wherein the thrust barrel moves linearly between a proximal position and a distal position for inserting the sensor with the skin-piercing device.

2. The analyte sensor applicator of claim 1, wherein the thrust barrel interfaces with the external cam surface through one or more grooves.

3. The analyte sensor applicator of claim 2, wherein upon activation of the cylindrical cam, the piston is pushed to the distal position by the thrust barrel and locked in the distal position and the thrust barrel returns to the proximal position without the piston.

4. The analyte sensor applicator of claim 1, wherein skin piercing device is configured to retract to the proximal position independent of the movement of the cylindrical cam.

5. The analyte sensor applicator of claim 2, wherein the cylindrical cam comprises one or more grooves on the external cam surface and the thrust barrel comprises one or more pins that ride in the grooves.

6. The analyte sensor applicator of claim 2, wherein the thrust barrel comprises one or more grooves on the external cam surface and the cylindrical cam comprises one or more pins that ride in the grooves.

7. The analyte sensor applicator of claim 2, wherein a speed of inserting the sensor is at least partially determined by a pattern of the one or more grooves.

8. The analyte sensor applicator of claim 1, wherein the analyte sensor applicator further comprises a spring positioned in contact with the cylindrical cam, the spring having a spring force that at least partially determines a speed of inserting the sensor.

9. The analyte sensor applicator of claim 1, wherein the piston comprises a piston base that engages with the wearable analyte monitoring device.

10. The analyte sensor applicator of claim 1, wherein the thrust barrel further comprises a magnetic component, the magnetic component magnetically engages with the wearable analyte monitoring device.

11. The analyte sensor applicator of claim 1, wherein the applicator housing comprises a housing bottom and wherein the analyte sensor applicator further comprises a sterile cap key configured to insert into an opening at a distal end of the housing bottom.

12. The analyte sensor applicator of claim 11, wherein the sterile cap key is configured to provide support to the wearable analyte monitoring device to keep the wearable analyte monitoring device in place.

13. The analyte sensor applicator of claim 11, wherein the analyte sensor applicator further comprises a sterile cap having a bottom portion and at least one tab, wherein the sterile cap key is configured to loosely engage with the sterile cap.

14. The analyte sensor applicator of claim 1, wherein the applicator further comprises an applicator frame having one or more slides on an internal surface of the applicator, the slide is aligned parallel to an axis of the applicator.

15. The analyte sensor applicator of claim 1, wherein the piston and/or the applicator housing comprises one or more vent holes.

16. The analyte sensor applicator of claim 14, wherein the piston comprises one or more anti-rotation features, each anti-rotation feature configured to move along one slide.

17. An implantation system comprising:
the analyte sensor applicator of claim 1; and
the wearable analyte monitoring device comprising:
an electronics assembly and sensor components coupled on a proximal side of the wearable analyte monitoring device; and
an adhesive component on a distal side of the wearable analyte monitoring device to adhere the wearable analyte monitoring device to a user,
wherein insertable portions of the skin-piercing device and the sensor project from the distal side.

18-34. (canceled)

35. An applicator system, comprising:
a housing comprising an opening at a bottom of the housing;
a wearable device configured to be housed in the housing; and
a key configured to be inserted into the housing through the opening,
wherein the key is in direct contact with the wearable device and provides support to the wearable device against a component of the applicator system.

36-48. (canceled)

49. An analyte senor for being implanted into a user, the analyte sensor comprising a sensing area, the sensing area configured to be placed inside a skin piercing device.
